(19) United States
(12) Patent Application Publication
(10) Pub. No.: US 2017/0361038 A1
(43) Pub. Date: Dec. 21, 2017

(54) Assembly for a Drug Delivery Device

(71) Applicant: Sanofi SA, Vernier (CH)

(72) Inventors: Stefan Mayer, Frankfurt am Main (DE); Steve Minshull, Cheshire (GB)

(21) Appl. No.: 15/542,939

(22) PCT Filed: Jan. 13, 2016
(86) PCT No.: PCT/EP2016/050571

§ 371 Date: Jul. 12, 2017

(30) Foreign Application Priority Data
Jan. 13, 2015 (EP) 15150964.3
Jan. 13, 2015 (EP) 15150970.0
Jan. 16, 2015 (EP) 15151509.5
Mar. 9, 2015 (EP) 15158270.7

(51) Int. Cl. A61M 15/00

(52) U.S. Cl. CPC A61M 15/0081; A61M 15/0071; A61M 15/0025; A61M 2205/273

(57) ABSTRACT

The present disclosure concerns an assembly for a drug delivery device (1)comprising a housing (3), a first abutment surface (64), a second abutment surface (21), and a detachable member (7) which is detachable from the housing (3) and attachable to the housing (3), wherein the assembly is configured such that, during detachment of the detachable member (7) from the housing (3) for an Nth time, N being a predetermined number, the first abutment surface (64) is brought into abutment with the second abutment surface (21), wherein the assembly is configured such that, when the first abutment surface (64) abuts with the second abutment surface (21), the detachable member (7) is enabled to be reattached to the housing (3) and, afterwards, the detachable member (7) is prevented from being detached from the housing (3).

This disclosure relates to an assembly for a drug delivery device. Furthermore, the disclosure relates to a drug delivery device comprising the assembly. The drug delivery device may be an inhalator, in particular a dry powder inhaler. An inhalation device is usually activated by a user's suction airstream and is intended for the inhalation of a substance, in particular a powdery substance. An inhalation device is described in document WO 2009/065707 A1, for example.

However, the assembly may also be suitable for other drug delivery devices, e.g. an injector such as an injection pen. In particular, the assembly may be used in a fixed-dose drug delivery device, i.e. a device in which the size of the dose of the drug which is delivered may not be varied by the user. Rather, in a fixed-dose device the size of the dose is set by the design of a dispensing mechanism.

It is an object of the present disclosure to provide an improved assembly for a drug delivery device, e.g. a mechanism preventing a further operation of the device after a given number of operation which requires only a low number of components.

This object may, inter alia, be achieved by the subject-matter of the independent claim. Advantageous embodiments and refinements are the subject-matter of the dependent claims. However, further advantageous concepts may be disclosed herein besides the ones which are claimed.

One aspect of the present disclosure relates to an assembly for a dry powder inhaler comprising a housing, a detachable member which is detachable from the housing and attachable to the housing, a mechanism configured to be transferred from one state to a subsequent state when the detachable member is detached from the housing.

The mechanism may be a step switching mechanism wherein the step switching mechanism comprises a finite number of states defining a chain of states such that the step switching mechanism is configured to be subsequently in each of the states of the chain of states, wherein the step switching mechanism is configured to be transferred from one state to a subsequent state of the chain of states when the detachable member is detached from the housing.

The dry powder inhaler may be a metering inhaler. A metering inhaler is designed to deliver a metered quantity of a drug with each dose delivery operation. The metering inhaler comprises a reservoir containing multiple doses of the metered quantity. The metering inhaler is designed such that the metered quantity is separated from the reservoir by the inhaler itself. Either the metered quantity may be separated from the reservoir in an initial part of each dose delivery operation or the metering quantity intended to be delivered in a subsequent dose delivery operation may be separated from the reservoir at a final part of a present dose delivery operation. The dry powder inhaler may be adapted for dispensing a plurality of doses of a drug or substance.

The mechanism may be defined by a first and a second abutment surface. In particular, each state of the step switching mechanism may correspond to a specific relative position between the first and the second abutment surface. With every detachment of the detachable member from the housing, the first and the second abutment surface may be moved closer together along a helical path.

The mechanism may be designed such that the first and the second abutment surface may only be moved into a specific relative position with respect to each other when they have passed a previous relative position with respect to each other, thereby defining the chain of states.

The finite number of states may correspond to a predetermined number N.

One aspect of the present disclosure relates to an assembly. The assembly may be suitable to be used in a lock-out mechanism for a drug delivery device. The device may be an inhalation device, in particular a dry powder inhalator. The device may be adapted for dispensing a plurality of doses of a medication or substance.

An assembly for a drug delivery device is provided which comprises a housing, a first abutment surface, a second abutment surface and a detachable member. The detachable member is detachable from the housing and attachable to the housing, wherein the assembly is configured such that, during detachment of the detachable member from the housing for an Nth time, N being a predetermined number, the first abutment surface is brought into abutment with the second abutment surface, wherein the assembly is configured such that, when the first abutment surface abuts with the second abutment surface, the detachable member is enabled to be reattached to the housing and, afterwards, the detachable member is prevented from being detached from the housing.

Accordingly, the assembly may be configured to ensure a lock-out of the drug delivery device after the detachable member has been detached from the housing N times and, afterwards, has been reattached to the housing. In particular, the assembly may be configured to prevent a user from trying to further use the device after the detachable member has been detached and reattached to the device N times.

The term “lock-out” may be understood in this context such that a further operation of the drug delivery device, e.g. a drug dispense operation, is prevented when the device is locked-out. The further operation may be prevented by the assembly in particular by preventing the detachment of the detachable member from the housing. A drug dispense operation may not be possible when the detachable member is attached to the housing.

Typically, every time the detachable member is detached from the device and afterwards reattached to the device, one drug dispense operation is carried out. In particular, the drug delivery device may be configured such that not more than one drug dispense operation can be carried out after the detachable member is detached from the device and before the detachable member is reattached to the device. Accordingly, the assembly may be configured to lock-out the drug delivery device after a maximum number of N drug delivery operations can be carried out.

The assembly may be an assembly for a lock-out mechanism configured to lock out the drug delivery device, i.e. to prevent a further operation of the device when the device is considered to be empty. The assembly may also be an assembly for a counter mechanism configured to count the number of doses left in the device or, alternatively, to count the number of possible drug dispense operations which may be carried before the device is considered to be empty wherein the counter-mechanism additionally has a lock-out feature preventing a further operation of the device when the device is considered to be empty.

The housing may be an external component of the drug delivery device. In particular, the housing may cover further elements of the drug delivery device.

The first abutment surface and the second abutment surface may be provided on different members of the drug delivery device. During normal operation of the drug delivery device, i.e. during a dose setting operation and/or a drug dispense operation before the device is considered to be empty, the first and the second abutment surface may be spaced apart from each other. The first and the second abutment surface may be configured to rotationally lock the a first member comprising the first abutment surface to each a second member comprising the second abutment surface when the first and the second abutment surface abut each other.

The detachable member being detachable from the housing may mean in this context that the detachable member can be completely removed from the housing such that it has no contact at all to the housing. When the detachable member is attached to the housing, the detachable member may cover at least a part of the housing. In particular, the detachable member may cover a part of the housing which comprises a mouthpiece such that no drug can be dispensed from the mouthpiece when the detachable member is attached to the housing. Thereby, the detachable member effectively prevents a drug dispense operation when attached to the housing. The detachable member may be a cap.

Attachment of the detachable member to the housing may be an operation which is carried out by a user. In this operation, the user may attach the previously detached detachable member to the housing, i.e. by screwing. Vice versa, detachment of the detachable member from the housing may be an operation wherein the detachable member is removed from the housing by the user, e.g. by screwing.

In one embodiment, the detachable member can be attached to the housing by screwing and detached from the housing by unscrewing. However, in alternative embodiments other means for attaching and detaching the detachable member are possible, e.g. the detachable member may be engageable with the housing via a bayonet connection or via a snap-fit mechanism.

The assembly provides the advantage of preventing a detachment from the housing with a minimal number of components. In particular, the first and the second abutment surface may be provided on members of the drug delivery device which are present in the drug delivery device anyway. Therefore, the assembly requires only minimal modifications of few elements of the drug delivery device and no further additional elements.

The design of the assembly ensuring that the detachable member can be reattached to the housing after it has been detached for the Nth time and is further prevented from being detached from the housing afterwards ensures that a user is warned that the device is considered to be empty. Thereby the user is prevented from trying to perform a drug delivery operation when no drug is left in the device.

The detachable member may be allowed to be rotated by a small angle relative to the housing when the detachable member is prevented from being detached from the housing. The small angle may be smaller than 10°, preferably smaller than 5°. The small angle may be in the range of 1° to 10°, preferably in the range of 1° to 5°. This feature may provide a very good warning to the user showing to the user that the operation of the drug delivery device is intentionally prevented. In particular, this design makes it obvious to the user that the drug delivery device is not damages as the detachable member is still movable by the small angle and, further, that the user is not supposed to operate the device any further as the range of motion of the detachable member has intentionally been limited to the small angle to indicate that the device is considered to be empty.

The detachable member may be rotatable relative to the housing by the small angle without damaging the detachable member or the housing. However, the detachable member may be prevented to be rotated by an angle bigger than the small angle relative to the housing in a way that such a rotation would result in a damaging of the detachable member and/or the housing.

In one embodiment, the first abutment surface is provided on a counting member comprising indicia and the second abutment surface is provided on a counter drive member. The counting member may be configured to follow a rotation of the counter drive member in the second rotational direction relative to the housing. The counting member may be prevented from following a rotation of the counter drive member in the first rotational direction relative to the housing.

In particular, a rotation of the counting member may increase or decrease a counted number. Further, the counter drive member may be configured to follow a rotation of the detachable member every time the detachable member is detached or attached to the housing. Thus, the assembly may be configured such that the counter drive member increases or decreases the counted number each time the detachable member is attached to the housing. The counter drive member may be constrained to rotate between a first and a second position. The first position of the counter drive member may corresponding to a configuration wherein the detachable member is attached to the housing. The second position of the counter drive member may correspond to a configuration wherein the detachable member is detached from the housing. Each time the detachable member alters its state from being attached to the housing to being detached from the housing, or vice versa, the counter drive member may be rotated.

In one embodiment, the counting member may be arranged such that one of the indicia is visible at any time through a window in the housing. Accordingly, this indicium may correspond to the currently counted number. As the counting member may be configured only to be rotated when the counter drive member rotates in the second rotational direction, the counting member may increase or decrease the counted number every time the counter drive member is moved from its second position to its first position. This may correspond to the counting member amending the counted number each time the detachable member is attached to the housing.

The predetermined number N may be determined by the position of the counting member relative to the housing. In particular, the predetermined number N may be determined by the initial position of the counting member relative to the housing, wherein the initial position is the position of the counting member relative to the housing after the assembly of the device is completed. This may be the position before the device is operated for the first time, i.e. before the first drug dispense operation is carried out. Accordingly, the assembly allows to set N to different values depending on the relative position of the counting member to the housing. Therefore, different drug delivery devices with different values of N may be constructed from the same components wherein the assembly process is modified in the respective relative position of the counting member to the housing to allow for different numbers N of possible operations.

The counter drive member and the counting member may be configured such that, when the first abutment surface abuts the second abutment surface, the counter drive member is prevented from rotating relative to the counting member.

The counter drive member may be rotated relative to the counting member during detachment of the detachable member from the housing. Further, the counter drive member may not be rotated relative to the counting member during attachment of the detachable member to the housing. Therefore, this design may ensure that the detachable member can be attached to the housing after it is detached for the Nth time and is further prevented from being detached from the housing afterwards.

The assembly may be configured such that, when the counter drive member is prevented from rotating relative to the counting member in the first rotational direction, the detachable member is prevented from rotation in the first rotational direction relative to the housing, thereby also being prevented from being detached from the housing.

Accordingly, the assembly is configured such that an abutment of two internal members, i.e. the counting member and the counter drive member, may prevent the detachment of the detachable member. As the counter drive member and the counting member are internal members, it is hardly possible for the user to override this mechanism preventing a detachment of the detachable member. Thus, the device is secure against operational mistakes of the user.

The drug delivery device may be configured such that a drug dispense operation is permitted only when the detachable member is detached from the housing. Accordingly, the abutment of the first and the second abutment surfaces may effectively prevent a further drug dispense operation.

The drug delivery device may be configured such that after a drug dispense operation has been carried out, the detachable member has to be reattached to the housing and, afterwards, the detachable member has to be detached from the housing to enable a subsequent drug dispense operation.

In particular, the detachable member may be configured to cover a mouthpiece of the drug delivery device when the detachable member is attached to the housing.

The detachable member may be configured such that, during detachment of the detachable member from the housing, the detachable member is rotated in a first rotational direction relative to the housing, wherein the detachable member is configured such that, during attachment of the detachable member to the housing, the detachable member is rotated in a second rotational direction relative to the housing which is opposite to the first rotational direction.

The detachable member may comprises a first projection and a second projection, wherein the counter drive member may comprise a protrusion, wherein the detachable member is configured such that, when the detachable member is rotated in the first rotational direction relative to the housing, the first projection engages the protrusion of the counter drive member such that the counter drive member follows the rotation of the detachable member in the first rotational direction, and wherein the detachable member is configured such that, when the detachable member is rotated in the second rotational direction relative to the housing, the second projection engage the protrusion of the counter drive member such that the counter drive member follows the rotation of the detachable member in the second rotational direction.

Accordingly, the counter drive member may be engaged with a different projection of the detachable member depending on the direction of the rotational movement of the detachable member. In particular, when the detachable member is first rotated in one direction and afterwards rotated in another rotational direction, the counter drive member may be disengaged from the detachable member for a short period of time wherein one of the projections is disengaged from the protrusion until the other of the projections engages the protrusion. This may be advantageous in case of a lock-out of the mechanism wherein the counter drive member may be prevented from moving and the detachable member is allowed to be rotated by a small angle before being prevented by the counter drive member from being rotated any further. As discussed above, this feature may provide a very good warning to the user showing to the user that the operation of the drug delivery device is intentionally prevented.

Further, the detachable member may be configured to engage with a metering rod during attachment of the detachable member to the housing. The assembly may be configured such that, during the attachment of the detachable member to the housing, the detachable member engages with the metering rod in a second relative position to said housing, and wherein said second relative position not being reachable during attachment before having passed a first relative position where a counted number is caused to be incremented or decremented either directly or during subsequent detachment. Thus, the assembly may be configured such that the detachable member engages with the metering rod after the incrementing or decrementing of a counted number has been initiated.

The engagement of the detachable member and the metering rod may be configured such that the metering rod follows a movement of the detachable member in the distal direction once the metering rod is engaged with the detachable member. Before engaging in this manner with each other, the detachable member may move the metering rod to its most proximal position during attachment of the detachable member. The engagement of the detachable member and the metering rod may be a snap-fit engagement.

In particular, the detachable member may comprise a snap-fit element that is configured to engage with the metering rod during attachment of the detachable member to the housing and that it is configured to be detached from the metering rod during detachment of the detachable member from the housing. The snap-fit element may force the metering rod to follow an axial movement of the detachable member when the snap-fit element is engaged with the metering rod.

In particular, the metering rod may be an element of a dose setting mechanism. The metering rod may have to be moved by the detachable member in the distal direction in order to set a subsequent dose of the drug. Thus, it may be ensured that it is impossible to set a subsequent dose, and thus to dispense a subsequent dose, after a drug dispense operation without previously incrementing or decrementing the counted number. The increment or decrement of the counted number may also correspond to a movement of the first and the second abutment surface closer to each other.

This design improves the safety of the assembly. In particular, this design may prevent a user from carrying out two subsequent drug dispense operations without incrementing or decrementing the counted number in between, i.e. without moving the first and the second abutment surface closer to each other. Accordingly, this design may ensure that it is not possible for the user to carry out more than N drug dispense operations. Accordingly, the safety of the device may be ensured by this feature as it is ensured that the lock-out of the mechanism cannot be prevented by the user. Thus, the user may be prevented from carrying out a drug dispense operation when the device is empty such that no drug or a too low dose of a drug would otherwise be dispensed.

The assembly may be configured such that during the attachment of the detachable member to the housing after the detachable member has been detached N times, the second relative position is not reachable during attachment before the detachable member has reached a configuration wherein it is no longer possible to detach the detachable member from the housing.

In particular, the assembly may be configured such that a projection projecting inwardly from the detachable member may engage with a corresponding outwardly protruding protrusion of a counter drive member before the detachable member engages with the metering rod in the second position during the attachment operation. The above-mentioned configuration may allow a movement of the detachable member relative to the housing but may prevent a detachment of the detachable member from the housing.

The detachable member may be allowed to be rotated by a small angle relative to the housing when the detachable member is prevented from being detached from the housing, and wherein the second relative position is within the small angle relative to the housing by which the detachable member is allowed to be rotated when the detachable member is prevented from being detached from the housing. This may ensure that the detachable member engages with the metering rod in a way that the metering rod follows a distal movement of the detachable member only after the counted number has been amended in the first N attachment operations. Further, this may ensure that the detachable member engages with the metering rod in a state wherein a detachment of the detachable member from the housing is no longer possible in the N+1th attachment operation. Accordingly, a user cannot manipulate the mechanism. In particular, it is not possible to remove a sub-quantity of the drug by partly attaching the detachable member and by, afterwards, removing the partly attached detachable member without incrementing or decrementing the counted number.

The detachable member may be configured to engage with the metering rod in such a manner that the metering rod removes a sub-quantity of a drug from a storage chamber during detachment of the detachable member from the housing, thereby setting the sub-quantity as a dose for the next drug dispense operation.

The metering rod may be configured to travel in the proximal direction during attachment of the detachable member from the housing. The metering rod may be in a most proximal position when the detachable member is fully attached to the housing. The detachable member may be configured to engage with the metering rod in a manner such that the metering rod follows a distal movement of the detachable member only when the metering rod is in its most proximal position.

The most proximal position of the metering rod may be a position wherein the metering rod has entered into the storage chamber. Accordingly, when the detachable member is fully attached to the housing, the metering rod may be in a position wherein it has entered into a storage chamber which comprises the drug. The assembly may be configured such that, after a drug dispense operation has been carried out, the metering rod has to be moved into its most proximal position to enable a subsequent drug dispense operation. Only when the metering rod has been moved into its most proximal position, the metering rod may be enabled to remove a sub-quantity of the drug from the storage chamber.

According to another aspect the present invention concerns a drug delivery device comprising the above-described assembly.

The assembly may be integrated in the device. In this way an improved, e.g. a compact and/or reliable and/or user-friendly, device may be achieved.

In the following text, a set of advantageous aspects is described. The aspects are numbered to facilitate referencing features of one aspect in other aspects. Features from the aspects are not only relevant in connection with the specific aspects they relate to but are also of relevance on their own.

1. An assembly for a drug delivery device comprising
a housing,
a first abutment surface,
a second abutment surface, and
a detachable member which is detachable from the housing and attachable to the housing,
wherein the assembly is configured such that, during detachment of the detachable
member from the housing for an Nth time, N being a predetermined number, the first abutment surface is brought into abutment with the second abutment surface,
wherein the assembly is configured such that, when the first abutment surface abuts with the second abutment surface, the detachable member is enabled to be reattached to the housing and, afterwards, the detachable member is prevented from being detached from the housing.

2. The assembly according to aspect 1,
wherein the detachable member is allowed to be rotated by a small angle relative to the housing when the detachable member is prevented from being detached from the housing.

3. The assembly according to one of the preceding aspects,
wherein the first abutment surface is provided on a counting member comprising indicia,
wherein the second abutment surface is provided on a counter drive member,
wherein the counting member is configured to follow a rotation of the counter drive member in the second rotational direction relative to the housing and wherein the counting member is prevented from following a rotation of the counter drive member in the first rotational direction relative to the housing.

4. The assembly according to aspect 3,
wherein the predetermined number N is determined by the position of the counting member relative to the housing.

5. The assembly according to one of aspects 3 or 4,
wherein the counter drive member and the counting member are configured such that, when the first abutment surface abuts the second abutment surface, the counter drive member is prevented from rotating relative to the counting member.

6. The assembly according to one of aspects 3 to 5,
wherein the assembly is configured such that, when the counter drive member is prevented from rotating relative to the counting member in the first rotational direction, the detachable member is prevented from rotation in the first rotational direction relative to the housing, thereby also being prevented from being detached from the housing.

7. The assembly according to one of the preceding aspects,
wherein the drug delivery device is configured such that a drug dispense operation is permitted only when the detachable member is detached from the housing.

8. The assembly according to one of the preceding aspects,
wherein the drug delivery device is configured such that, after a drug dispense operation has been carried out, the detachable member has to be reattached to the housing and, afterwards, the detachable member has to be detached from the housing to enable a subsequent drug dispense operation.

9. The assembly according to one of the preceding aspects,
wherein the detachable member is configured to cover a mouthpiece of the drug delivery device when the detachable member is attached to the housing.

10. The assembly according to one of the preceding aspects,
wherein the detachable member is configured such that, during detachment of the detachable member from the housing, the detachable member is rotated in a first rotational direction relative to the housing,
wherein the detachable member is configured such that, during attachment of the detachable member to the housing, the detachable member is rotated in a second rotational direction relative to the housing which is opposite to the first rotational direction.

11. The assembly according to one of the preceding aspects,
wherein the detachable member comprises a first projection and a second projection,
wherein the counter drive member comprises a protrusion,
wherein the detachable member is configured such that, when the detachable member is rotated in the first rotational direction relative to the housing, the first projection engages the protrusion of the counter drive member such the counter drive member follows the rotation of the detachable member in the first rotational direction, and
wherein the detachable member is configured such that, when the detachable member is rotated in the second rotational direction relative to the housing, the second projection engages the protrusion of the counter drive member such the counter drive member follows the rotation of the detachable member in the second rotational direction.

12. The assembly according to one of the preceding aspects,
wherein the detachable member is configured to engage with a metering rod during attachment of the detachable member to the housing, and
wherein the assembly is configured such that, during the attachment of the detachable member to the housing, the detachable member engages with the metering rod in a second relative position to said housing, and wherein said second relative position not being reachable during attachment before having passed a first relative position where a counted number is caused to be incremented or decremented either directly or during subsequent detachment.

13. The assembly according to aspect 12,
wherein the assembly is configured such that during the attachment of the detachable member to the housing after the detachable member has been detached N times, the second relative position is not reachable during attachment before the detachable member has reached a configuration wherein it is no longer possible to detach the detachable member from the housing.

14. The assembly according to aspect 12 or 13,
wherein the detachable member is allowed to be rotated by a small angle relative to the housing when the detachable member is prevented from being detached from the housing, and
wherein the second position of the detachable member relative to the housing is within the small angle relative to the housing by which the detachable member is allowed to be rotated when the detachable member is prevented from being detached from the housing.

15. The assembly according to one of aspects 12 to 14,
wherein the detachable member is configured to engage with the metering rod in such a manner that the metering rod removes a sub-quantity of a drug from a storage chamber during detachment of the detachable member from the housing, thereby setting the sub-quantity as a dose for the next drug dispense operation.

16. The assembly according to one of aspects 12 to 15,
wherein the metering rod is configured to travel in the proximal direction during attachment of the detachable member to the housing,
wherein the metering rod is in a most proximal position when the detachable member is fully attached to the housing,
wherein the detachable member is configured to engage with the metering rod in a manner such that the metering rod follows a distal movement of the detachable member only when the metering rod is in its most proximal position.

17. Drug delivery device comprising an assembly according to one of the preceding aspects.

Another aspect of the present disclosure related to an improved assembly for a drug delivery device, e.g. a mechanism allowing to count a counted number requiring a low number of components. This aspect of the present disclosure relates to an assembly.

This aspect may be present in an assembly for a drug delivery device, in particular in an assembly for a dry powder inhaler or a metering inhaler, additionally or alternatively to the above discussed aspects.

The assembly according to this aspect may be suitable to be used in a counter mechanism for a drug delivery device. The device may be an inhalation device, in particular a dry powder inhalator. The device may be adapted for dispensing a plurality of doses of a drug or substance.

An assembly for a drug delivery device is provided which comprises a housing, a counter drive member configured to increase or decrease a counted number when the counter drive member is rotated in a second rotational direction relative to the housing, and a detachable member which is detachable from the housing and attachable to the housing, wherein the detachable member is configured such that, during detachment of the detachable member from the housing, the detachable member is rotated in a first rotational direction relative to the housing and the counter drive member follows the rotation of the detachable member in the first rotational direction, and wherein the detachable member is configured such that during attachment of the detachable member to the housing the detachable member is rotated in the second rotational direction relative to the housing which is opposite to the first rotational direction and the counter drive member follows the rotation of the detachable member in the second rotational direction.

Accordingly, the assembly may be configured such that the counter drive member increases or decreases the counted number each time the detachable member is attached to the housing. The assembly may be configured to provide information regarding the number of attachment processes carried out. In particular, each time the detachable member is detached and afterwards reattached to the housing, a drug dispense operation may be performed. Therefore, the counted number may correspond to the number of drug dispense operations which have been carried out or, alternatively, the counted number may correspond to the number of drug dispense operations which are still possible before the device is considered to be empty.

The drug delivery device may be configured such that after detachment of the detachable member and before reattachment of the detachable member to the housing, at the most one drug dispense operation can be carried out. Accordingly, if the counted number is increased or decreased every time the detachable member is attached, the counter drive member may provide information about the minimum number of possible drug dispense operations. In this case, the counted number may corresponds to the minimum number of doses left in the device.

The assembly may be an assembly for a counter mechanism configured to count the number of doses left in the device or, alternatively, to count the number of possible drug dispense operations which may be carried out before the device is considered to be empty.

The housing may be an external component of the drug delivery device. In particular, the housing may cover further elements of the drug delivery device.

The counter drive member may be configured to interact with further elements of a counting mechanism, e.g. with a counting member comprising indicia. The indicia may be numbers printed on an outer surface of the counting member.

In an alternative design, the counter drive member may itself comprise indicia and may be configured to display the counted number.

The counter drive member may be constrained to rotate between a first and a second position. The first position of the counter drive member may correspond to a configuration wherein the detachable member is attached to the housing. The second position of the counter drive member may correspond to a configuration wherein the detachable member is detached from the housing. Each time the detachable member alters its state from being attached to the housing to being detached from the housing or vice versa, the counter drive member may be moved. In particular, the counter drive member may follow a rotation of the detachable member in this situation.

The counter drive member may be configured to trigger a counting of a counting member, i.e. to trigger an increase or a decrease of the counted number. The counter drive member may be configured to transfer a movement of the detachable member into a movement of the counting member.

The detachable member being detachable from the housing may mean in this context that the detachable member can be completely removed from the housing such that it has no contact at all to the housing. When the detachable member is attached to the housing, the detachable member may cover at least a part of the housing. In particular, the detachable member may cover a part of the housing which comprises a mouthpiece such that no drug can be dispensed from the mouthpiece when the detachable member is attached to the housing. Thereby, the detachable member may effectively prevent a drug dispense operation when attached to the housing. The detachable member may be a cap.

Attachment of the detachable member to the housing may be an operation which is carried out by a user. In this operation, the user may attach the previously detached detachable member to the housing, e.g. by screwing. Vice versa, detachment of the detachable member from the housing may be an operation wherein the detachable member is removed from the housing by the user, e.g. by screwing.

In one embodiment, the detachable member can be attached to the housing by screwing and detached from the housing by unscrewing. However, in alternative embodiments other means for attaching and detaching the detachable member are possible, e.g. the detachable member may be engageable with the housing via a bayonet connection or via a snap-fit mechanism.

The assembly provides the advantage of allowing the counting of a counted number with a minimal number of components. In particular, only the counter drive member and the counting member are required as additional components compared to a drug delivery device not comprising a counting mechanism. Moreover, this design of the counting mechanism requires only minimal modifications of other elements of the drug delivery device.

The counter drive member may be constrained such that it can only carry out a helical movement relative to the housing. The detachable member may be constrained such that it can only out a helical movement relative to the housing. The counter drive member and the detachable member may be configured such that, when the counter drive member follows a rotational movement of the detachable member, the counter drive member carries out a helical movement with an axial component being directed opposite to an axial component of the helical movement carried out by the detachable member.

Each helical movement comprises a rotational component and an axial component. The detachable member may be constrained in its movement to carry out a helical movement relative to the housing only when the detachable member is in contact to the housing. After the detachable member is completely detached from the housing, the detachable member may be free to carry out any arbitrary movement relative to the housing. However, during attachment to and detachment from the housing, the detachable member may be constrained such that it can only carry out a helical movement relative to the housing of the drug delivery device.

As the counter drive member and the detachable member may move in different axial directions when the counter drive member follows a rotational movement of the detachable member, they may be designed such that they are disengaged at a predefined point during this rotational movement of the detachable member. Further, the axial component of the helical movement carried out by the counter drive member and the axial component of the helical movement carried out by the detachable member may differ in their amplitude. The amplitude of the respective axial component may be defined by a slope of a thread along which the respective element runs.

The counter drive member may comprise a protrusion. The detachable member may comprise a first projection and a second projection. The detachable member may be configured such that, when the detachable member rotates in the first rotational direction, the first projection engages the protrusion of the counter drive member such that the counter drive member follows the rotation of the detachable member in the first rotational direction. The detachable member may be configured such that, when the detachable member rotates in the second rotational direction, the second projection engages the protrusion of the counter drive member such that the counter drive member follows the rotation of the detachable member in the second rotational direction.

Accordingly, the counter drive member may be engaged with a different projection of the detachable member depending on the direction of the rotational movement of the detachable member. In particular, when the detachable member is first rotated in one direction and afterwards rotated in another rotational direction, the counter drive member may be disengaged from the detachable member for a short period of time wherein one of the projections is disengaged from the protrusion until the other of the projections engages the protrusion. This may be advantageous in case of a lock-out of the mechanism wherein the counter drive member may be prevented from moving and the detachable member is allowed to be rotated by a small angle before being blocked by the counter drive member from being rotated any further. The small angle may be in the range of 1° to 10°, preferably in the range of 1° to 5°. This feature may provide a very good warning to the user showing to the user that the operation of the drug delivery device is intentionally prevented.

The protrusion of the counter drive member be inclined relative to an azimuthal direction. The azimuthal direction is a direction defined by a circle around a rotational axis of the drug delivery device wherein the circle lies in a plane that is perpendicular to the rotational axis of the drug delivery device.

In particular, when the counter drive member is constrained to carry out a helical movement, the protrusion may slide along a correspondingly inclined ramp of the housing, thereby allowing the counter drive member to move along its helical path relative to the housing with a minimal friction. The angle of inclination may define the amplitude of the axial component of a helical movement carried out by the counter drive member.

The first and the second projection of the detachable member may be spaced apart in the azimuthal and in the axial direction. This design may ensure that the first projection engages with the protrusion of the counter drive member during detachment of the detachable member and that the second projection engages with the counter drive member during attachment of the detachable member. The projections may be arranged at an inner surface of the detachable member.

The housing may comprise a cut-out. The counter drive member may be arranged at least partially inside the housing. The detachable member may be arranged outside of the housing. The protrusion of the counter drive member may protrude through the cut-out of the housing.

The cut-out of the housing may have an inclined form. Thus, if the protrusion is also inclined relative to the azimuthal direction, the protrusion and the inclined cut-out are configured to interact with each other with a minimal friction wherein the cut-out forces the counter drive member to travel along a helical path when rotated relative to the housing. As the protrusion of the counter drive member may protrude through the cut-out of the housing, the counter drive member may be allowed to interact with the detachable member which may be arranged outside of the housing.

During the detachment of the detachable member from the housing, the detachable member may be configured to carry out a detachment movement comprising a first part wherein the detachable member rotates in the first rotational direction relative to the housing and a subsequent second part wherein the detachable member rotates in the first rotational direction relative to the housing. The detachable member and the counter drive member may be configured such that, during detachment of the detachable member from the housing, the counter drive member follows only the first part of the detachment movement of the detachable member. The first part may be an initial part of the detachment movement.

Further, during the attachment of the detachable member to the housing, the detachable member may be configured to carry out an attachment movement comprising a first part wherein the detachable member rotates in the second rotational direction relative to the housing and a subsequent second part wherein the detachable member rotates in the second rotational direction relative to the housing. The detachable member and the counter drive member may be configured such that, during attachment of the detachable member to the housing, the counter drive member follows only the second part of the attachment movement of the detachable member. The second part may be a final part of the attachment movement.

The counter drive member may comprise a spring element biasing the counter drive member in an axial direction. This may ensure that the counter drive member stays in its second position when the detachable member is detached from the housing.

The counter drive member may comprise a slot in the axial direction. The slot may ensure that the counter drive member can be compressed during a manufacturing process such that it can be inserted into the housing. Once the manufacturing is completed, the counter drive member may be allowed to release its tension and to extend in the radial direction, thereby enlarging the slot and engaging properly with the housing. Accordingly, the counter drive member may be a C-shaped element.

The assembly may further comprise a counting member having indicia which correspond to the counted number. The counting member may be configured to follow a rotation of the counter drive member in the second rotational direction relative to the housing. The counting member may be prevented from following a rotation of the counter drive member in the first rotational direction relative to the housing.

In one embodiment, the counting member may be arranged such that one of the indicia is visible at any time through a window in the housing. Accordingly, this one of the indicia may correspond to the currently counted number. As the counting member may be configured only to be rotated when the counter drive member rotates in the second rotational direction, the counting member may increase or decrease the counted number every time the counter drive member is moved from its second position to its first position. This may correspond to the counting member amending the counted number each time the detachable member is attached to the housing.

The counting member may be constrained such that it can only carry out a helical movement relative to the housing of the drug delivery device. The counting member may be configured such that the helical movement has an axial component in the distal direction. Accordingly, the counting member may be move closer towards the distal end of the drug delivery device every time the detachable member is attached.

The counting member may be arranged inside the housing. The housing may comprise an inner surface having axially extending ribs. The counting member may comprise a claw being engaged with the axially extending ribs. The axially extending ribs of the housing and the claw of the counting member may be configured to allow a rotation of the counting member in the second rotational direction relative to the housing and to prevent a rotation of the counting member in the first rotational direction relative to the housing.

For this purpose, the claw and the axially extending ribs may comprise correspondingly tapered surfaces. This configuration may prevent that the counting member is rotated in the first rotational direction, thereby unintentionally amending the counted number.

The assembly may be configured such that the drug dispense operation is permitted only when the detachable member is detached from the housing. The assembly may be configured such that, after a drug dispense operation has been carried out, the detachable member has to be reattached to the housing and, afterwards, the detachable member has to be detached from the housing to enable a subsequent drug dispense operation. This design may ensure that the counted number is incremented or decremented at least each time a drug dispense operation is carried out. In particular, the counter drive member may rotate in the second rotational direction every time the detachable member is attached to the housing.

In the following text, a set of advantageous aspects is described. The aspects are numbered to facilitate referencing features of one aspect in other aspects. Features from the aspects are not only relevant in connection with the specific aspects they relate to but are also of relevance on their own.

1. Assembly for a drug delivery device, comprising
a housing,
a counter drive member configured to increase or decrease a counted number when the counter drive member is rotated in a second rotational direction relative to the housing, and
a detachable member which is detachable from the housing and attachable to the housing,
wherein the detachable member is configured such that, during detachment of the detachable member from the housing, the detachable member is rotated in a first rotational direction relative to the housing and the counter drive member follows the rotation of the detachable member in the first rotational direction, and
wherein the detachable member is configured such that, during attachment of the detachable member to the housing, the detachable member is rotated in the second rotational direction relative to the housing which is opposite to the first rotational direction and the counter drive member follows the rotation of the detachable member in the second rotational direction.

2. Assembly according to aspect 1,
wherein the counter drive member is constrained such that it can only carry out a helical movement relative to the housing,
wherein the detachable member is constrained such that it can only carry out a helical movement relative to the housing, and
wherein the counter drive member and the detachable member are configured such that, when the counter drive member follows a rotational movement of the detachable member, the counter drive member carries out a helical movement with an axial component being directed opposite to an axial component of the helical movement carried out by the detachable member.

3. Assembly according to one of the preceding aspects,
wherein the counter drive member comprises a protrusion,
wherein the detachable member comprising a first projection and a second projection, and
wherein the detachable member is configured such that, when the detachable member rotates in the first rotational direction, the first projection engages the protrusion of the counter drive member such the counter drive member follows the rotation of the detachable member in the first rotational direction, and
wherein the detachable member is configured such that, when the detachable member rotates in the second rotational direction, the second projection engages the protrusion of the counter drive member such that the counter drive member follows the rotation of the detachable member in the second rotational direction.

4. Assembly according aspect 3,
wherein the protrusion of the counter drive member is inclined relative to an azimuthal direction.

5. Assembly according to one of aspects 3 or 4,
wherein the first and the second projection of the detachable member are spaced apart in an azimuthal direction and in an axial direction.

6. Assembly according to one of aspects 3 to 5,
wherein the housing comprises a cut-out,
wherein the counter drive member is arranged at least partially inside the housing,
wherein the detachable member is arranged outside of the housing, and
wherein the protrusion of the counter drive member protrudes through the cut-out of the housing.

7. Assembly according to one of the preceding aspects,
wherein, during the detachment of detachable member from the housing, the detachable member is configured to carry out a detachment movement comprising a first part
wherein the detachable member rotates in the first rotational direction relative to the housing and a subsequent second part wherein the detachable member rotates in the first rotational direction relative to the housing,
wherein the detachable member and the counter drive member are configured such that, during detachment of the detachable member from the housing, the counter drive member follows only the first part of the detachment movement of the detachable member.

8. Assembly according to one of the preceding aspects,
wherein, during the attachment of detachable member to the housing, the detachable member is configured to carry out an attachment movement comprising a first part
wherein the detachable member rotates in the second rotational direction relative to the housing and a subsequent second part wherein the detachable member rotates in the second rotational direction relative to the housing,
wherein the detachable member and the counter drive member are configured such that, during attachment of the detachable member to the housing, the counter drive member follows only the second part of the attachment movement of the detachable member.

9. Assembly according to one of the preceding aspects,
wherein the counter drive member comprises a spring element biasing the counter drive member in an axial direction.

10. Assembly according to one of the preceding aspects,
wherein the counter drive member comprises a slot in an axial direction.

11. Assembly according to one of the preceding aspects,
further comprising a counting member having indicia which correspond to the counted number,
wherein the counting member is configured to follow a rotation of the counter drive member in the second rotational direction relative to the housing, and
wherein the counting member is prevented from following a rotation of the counter drive member in the first rotational direction relative to the housing.

12. Assembly according to aspect 11,
wherein the counting member is constrained such that it can only carry out a helical movement relative to the housing.

13. Assembly according to one of aspects 11 or 12,
wherein the counting member is arranged inside the housing,
wherein the housing comprises an inner surface having axially extending ribs,
wherein the counting member comprises a claw being engaged with the axially extending ribs, and
wherein the axially extending ribs of the housing and the claw of the counting member are configured to allow a rotation of the counting member in the second rotational direction relative to the housing and to prevent a rotation of the counting member in the first rotational direction relative to the housing.

14. The assembly according to one of the preceding aspects,
wherein the assembly is configured such that a drug dispense operation is permitted only when the detachable member is detached from the housing, and
wherein the assembly is configured such that, after a drug dispense operation has been carried out, the detachable member has to be reattached to the housing and, afterwards, the detachable member has to be detached from the housing to enable a subsequent drug dispense operation.

15. Drug delivery device comprising an assembly according to one of the preceding aspects.

The term “drug” or “substance”, as used herein may mean a pharmaceutical formulation containing at least one pharmaceutically active compound, for example for the treatment of obstructive airway or lung diseases such as asthma or chronic obstructive pulmonary disease (COPD), local respiratory tract oedema, inflammation, viral, bacterial, mycotic or other infection, allergies, diabetes mellitus.

The active pharmaceutical compound is preferably selected from the group consisting of active pharmaceutical compounds suitable for inhalation, preferably antiallergenic, antihistamine, anti-inflammatory, antitussive agents, bronchodilators, anticholinergic drugs, and combinations thereof.

The active pharmaceutical compound may for example be chosen from:

an insulin such as human insulin, e.g. a recombinant human insulin, ora human insulin analogue or derivative, a glucagon-like peptide (GLP-1) or an analogue or derivative thereof, or exendin-3 or exendin-4 or an analogue or derivative of exendin-3 or exendin-4;

an adrenergic agent such as a short acting β2-agonists (e.g. Salbutamol, Albuterol, Levosalbutamol, Fenoterol, Terbutaline, Pirbuterol, Procaterol, Bitolterol, Rimiterol, Carbuterol, Tulobuterol, Reproterol), a long acting β2-agonist (LABA, e.g. Arformoterol, Bambuterol, Clenbuterol, Formoterol, Salmeterol), an ultra LABA (e.g. Indacaterol) or another adrenergic agent (e.g. Epinephrine, Hexoprenaline, Isoprenaline (Isoproterenol), Orciprenaline (Metaproterenol));

a glucocorticoid (e.g. Beclometasone, Budesonide, Ciclesonide, Fluticasone, Mometasone, Flunisolide, Betamethasone, Triamcinolone);

an anticholinergic agent or muscarinic antagonist (e.g. Ipratropium bromide, Oxitropium bromide, Tiotropium bromide);

a mast cell stabilizer (e.g. Cromoglicate, Nedocromil);

a xanthine derivative (e.g. Doxofylline, Enprofylline, Theobromine, Theophylline, Aminophylline, Choline theophyllinate);

an eicosanoid inhibitor, such as a leukotriene antagonist (e.g. Montelukast, Pranlukast, Zafirlukast), a lipoxygenase inhibitor (e.g. Zileuton) or a thromboxane receptor antagonist (e.g. Ramatroban, Seratrodast);

a phosphodiesterase type-4 inhibitor (e.g. Roflumilast);

an antihistamine (e.g. Loratadine, Desloratadine, Cetirizen, Levocetirizine, Fexofenadine);

an allergen immunotherapy (e.g. Omalizumab);

a mucolytic (e.g. Carbocisteine, Erdosteine, Mecysteine); an antibiotic or antimycotic;

or a combination of any two, three or more of the above-mentioned compound classes or compounds (e.g. Budesonide/Formoterol, Fluticasone/Salmeterol, Ipratropium bromide/Salbutamol, Mometasone/Formoterol);

or a pharmaceutically acceptable salt or solvate or esters of any of the above named compounds.

Pharmaceutically acceptable salts are for example acid addition salts and basic salts. Acid addition salts are e.g. a chloride, bromide, iodide, nitrate, carbonate, sulfate, methylsulfate, phosphate, acetate, benzoate, benzenesulfonate, fumarate, malonate, tartrate, succinate, citrate, lactate, gluconate, glutamate, edetate, mesylate, pamoate, pantothenate or a hydroxy-naphthoate salt. Basic salts are for example salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology. Pharmaceutically acceptable ester may for example be acetates, propionates, phosphates, succinates or etabonates.

Pharmaceutically acceptable solvates are for example hydrates.

Further features and refinements become apparent from the following description of the exemplary embodiments in connection with the accompanying figures.

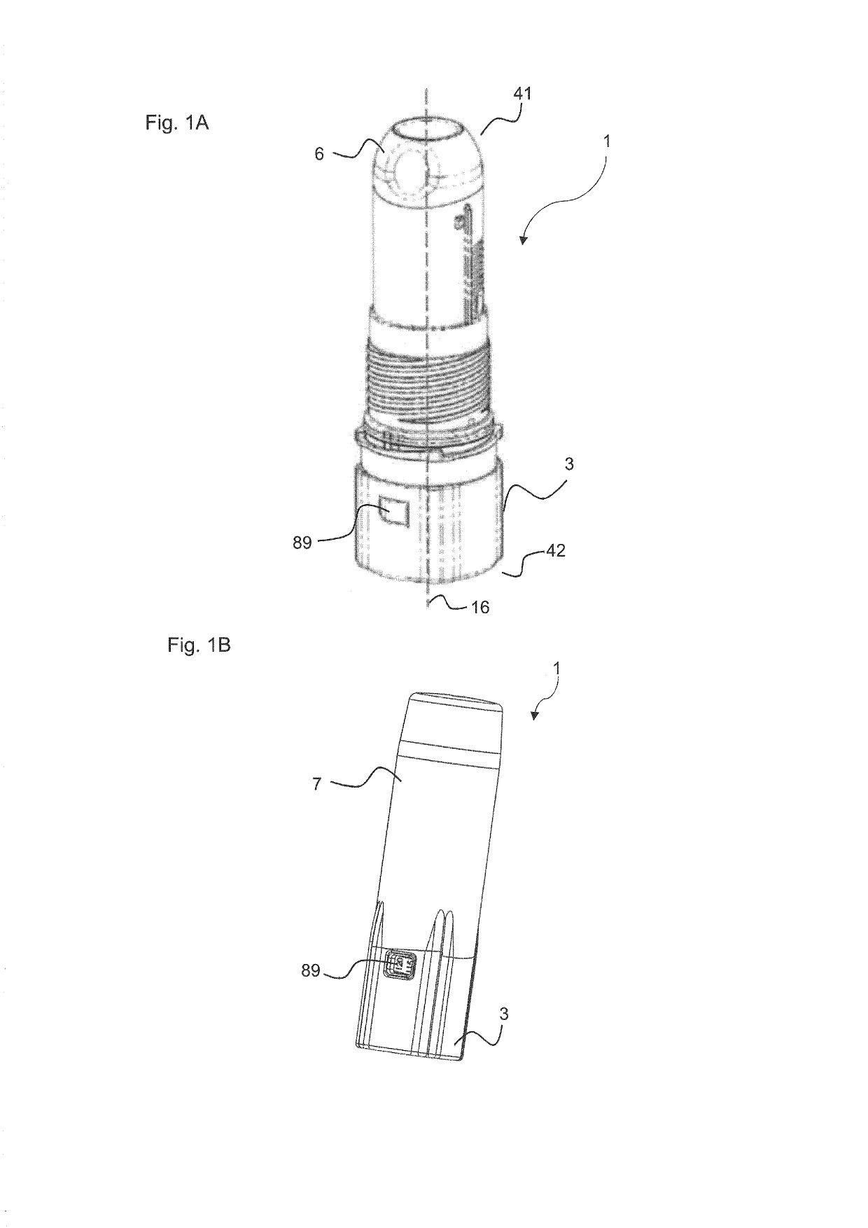

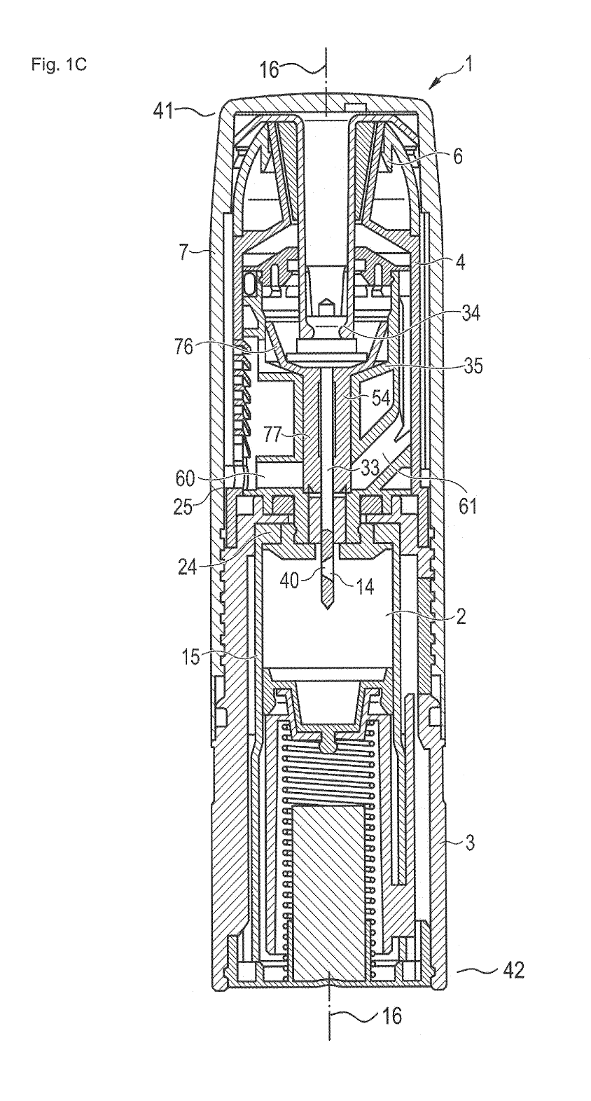

FIGS. 1A and 1B schematically show a perspective view of a drug delivery device,

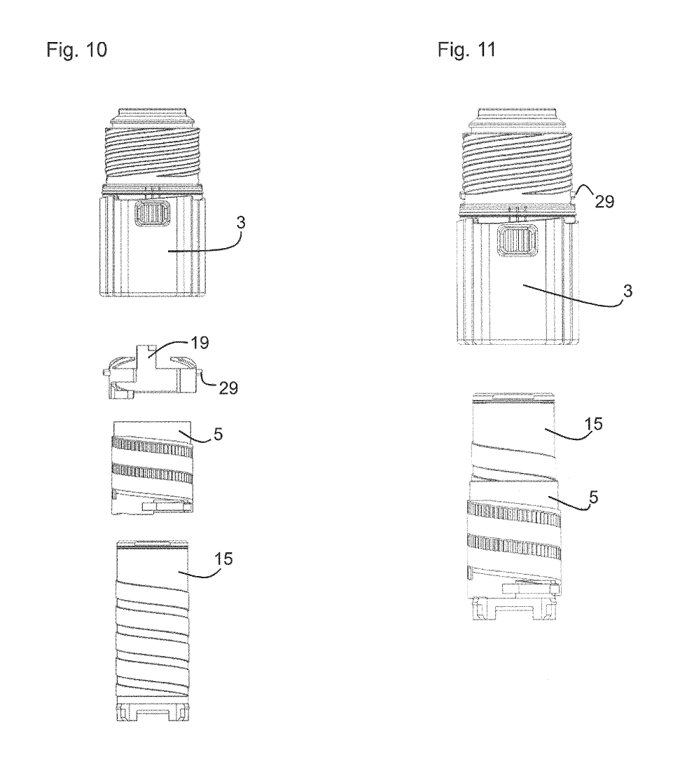

FIG. 10 schematically shows a sectional side view of the drug delivery device of FIGS. 1A and 1B,

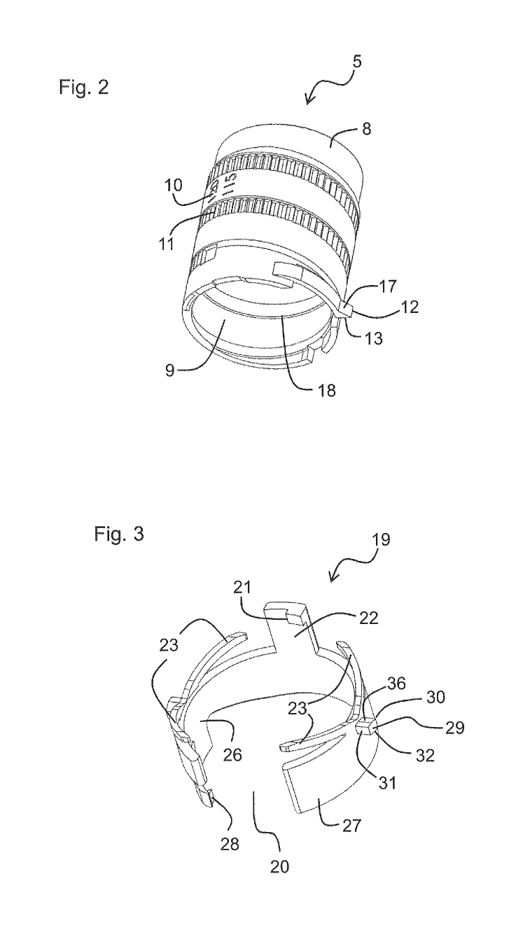

FIG. 2 shows a perspective view of a counting member,

FIG. 3 shows a perspective view of a counter drive member,

FIG. 4 shows a cross-sectional view of a detachable member,

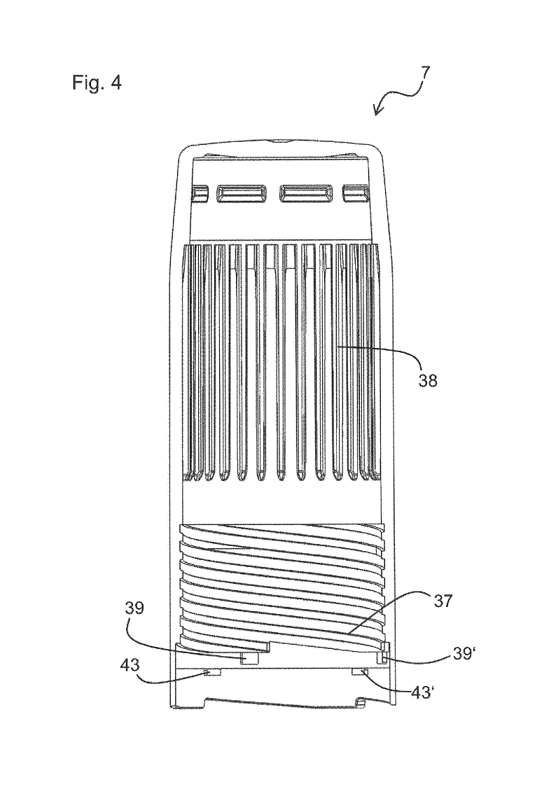

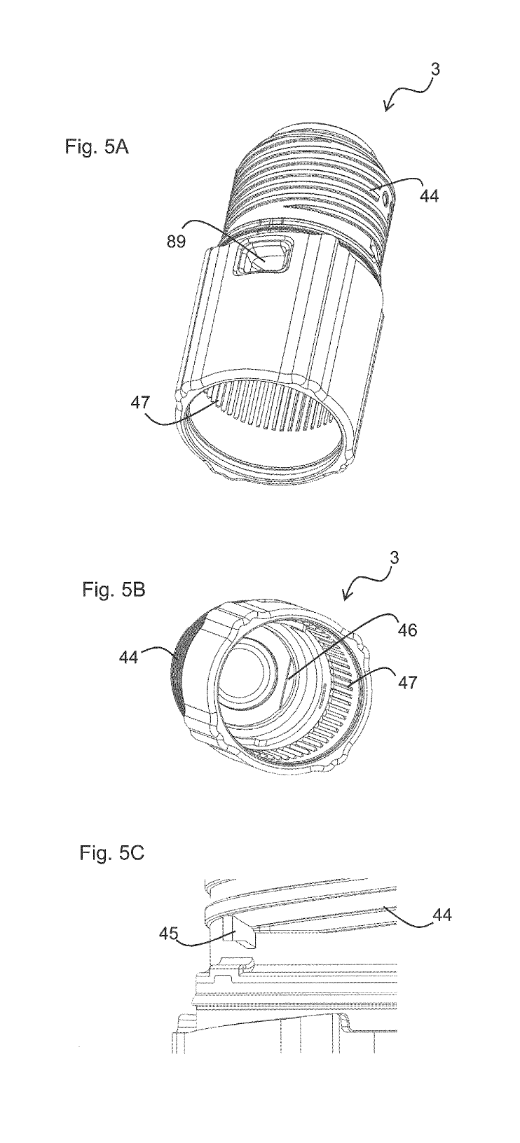

FIG. 5A and FIG. 5B show perspective views of a housing,

FIG. 5C shows an enlarged view of a part of the housing,

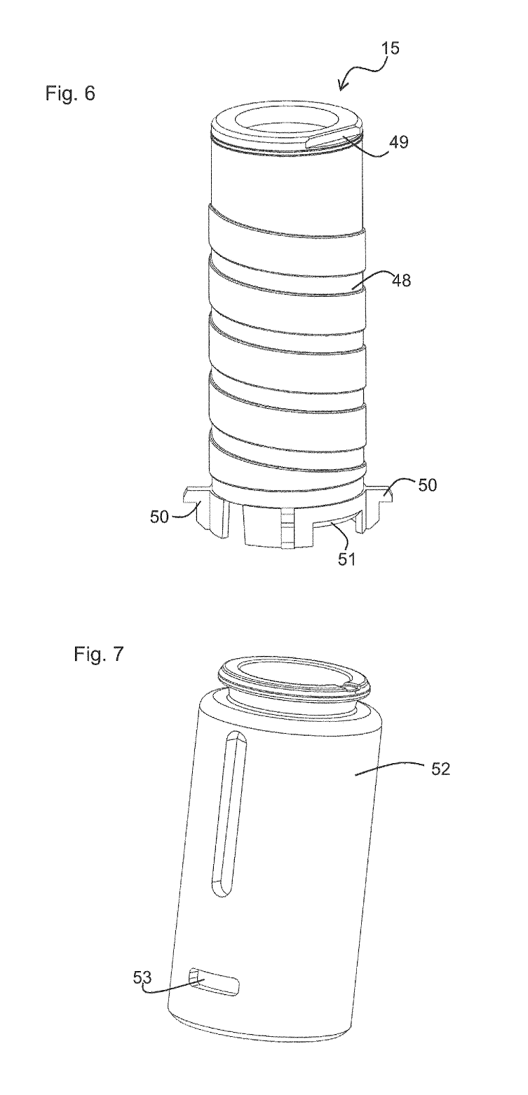

FIG. 6 shows a perspective view of a storage chamber,

FIG. 7 shows a perspective view of a spring housing,

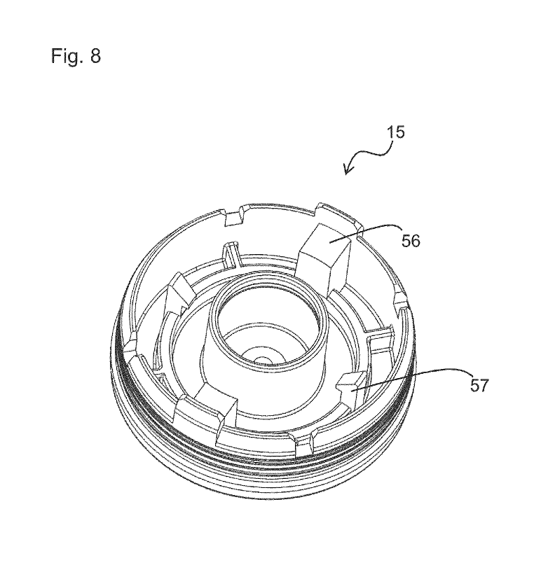

FIG. 8 shows a perspective view of a bottom lid,

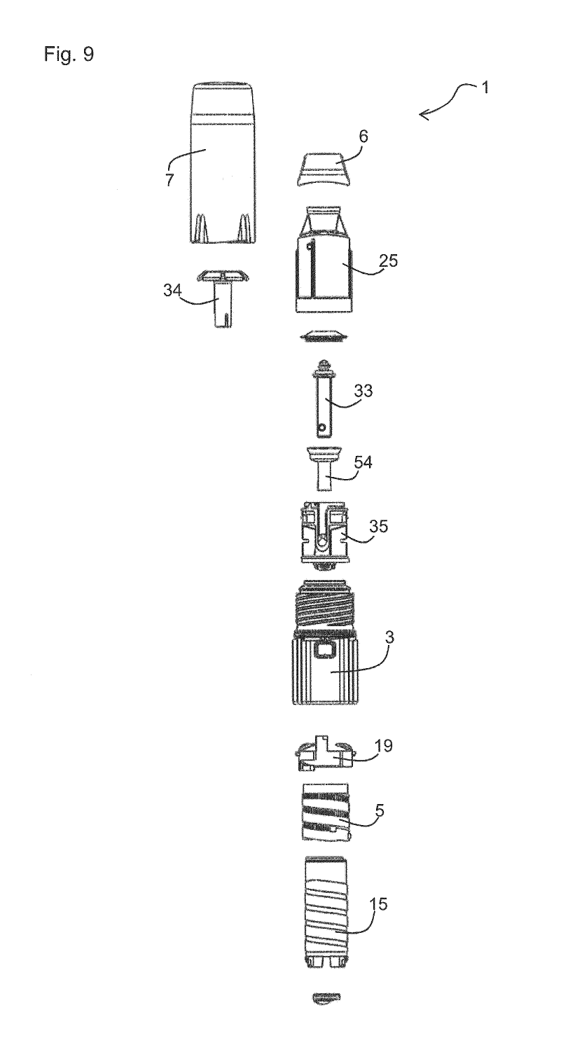

FIG. 9 shows an exploded view of the drug delivery device,

FIGS. 10, 11 and 12 indicate the assembly process of the device,

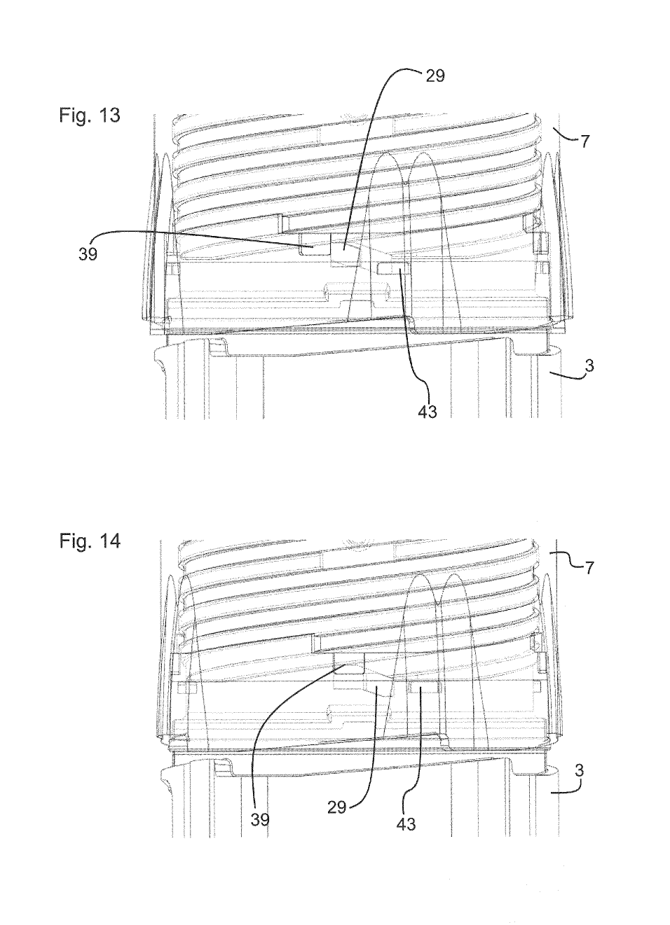

FIG. 13 shows an enlarged view of a part of the device in a state wherein the detachable member is completely attached to the housing,

FIG. 14 shows an enlarged view of a part of the device in a state wherein the detachable member is partly detached from the housing,

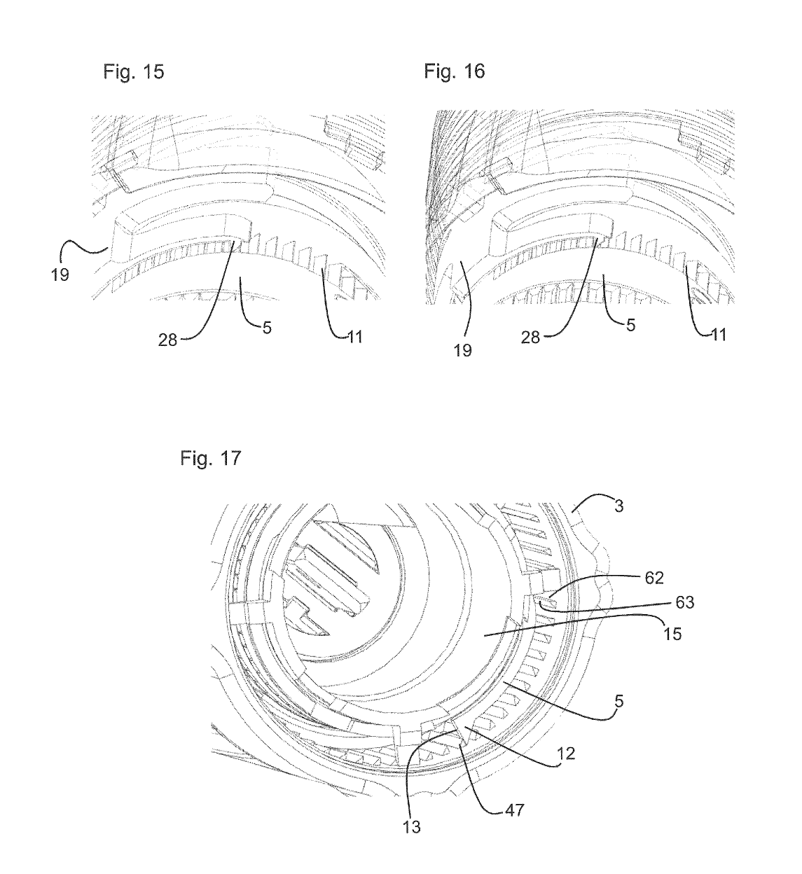

FIGS. 15 and 16 show details of the interaction of the counter drive member and the counting member during the detachment of the detachable member,

FIG. 17 shows details of the interaction of the counting member and the housing,

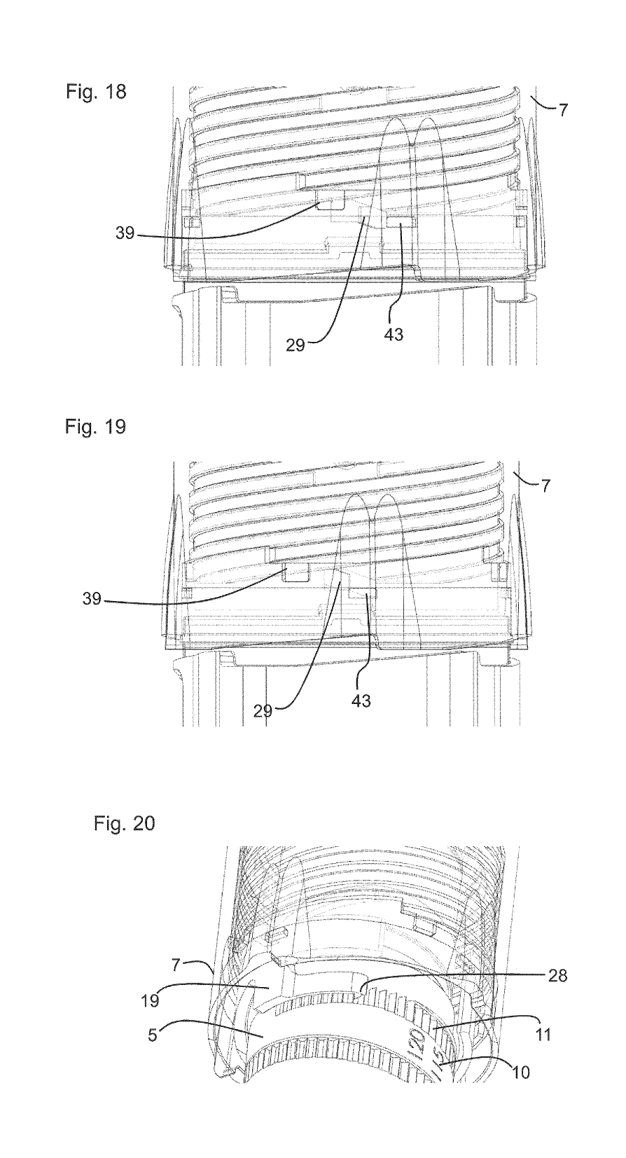

FIG. 18 shows a detailed view of a part of the device at the end of the first part of the attachment movement,

FIG. 19 shows a detailed view of a part of the device at the end of the attachment movement,

FIG. 20 shows the interaction of the counter drive member and the counting member during the attachment of the attachable member to the housing,

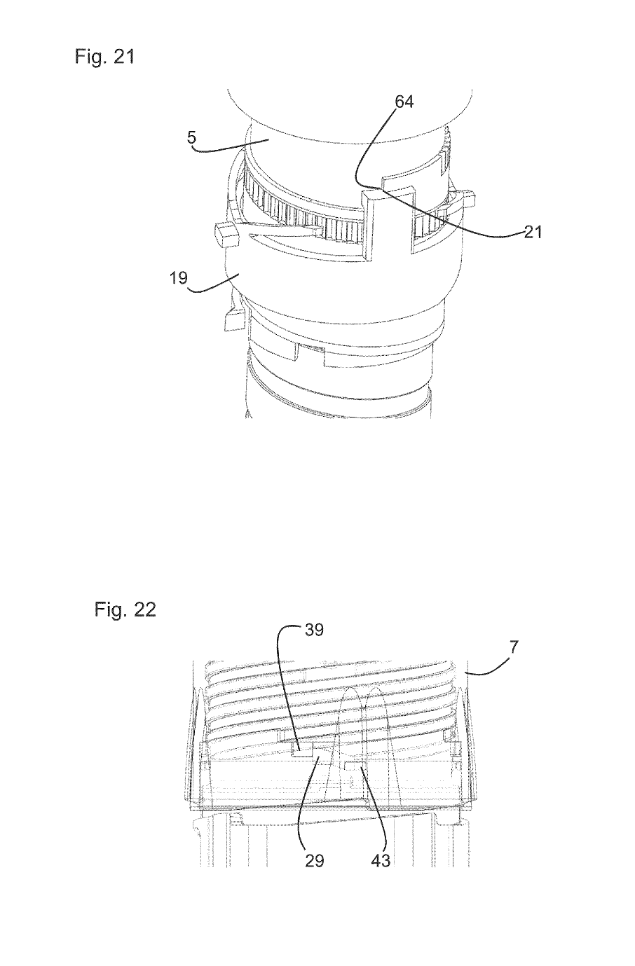

FIG. 21 shows a perspective view of the interaction of the counter drive member and the counting member when the detachable member has been detached N times,

FIG. 22 shows the interaction of the counter drive member and the detachable member in a configuration wherein the detachable member has been detached N times and, afterwards, has been reattached to the housing, and

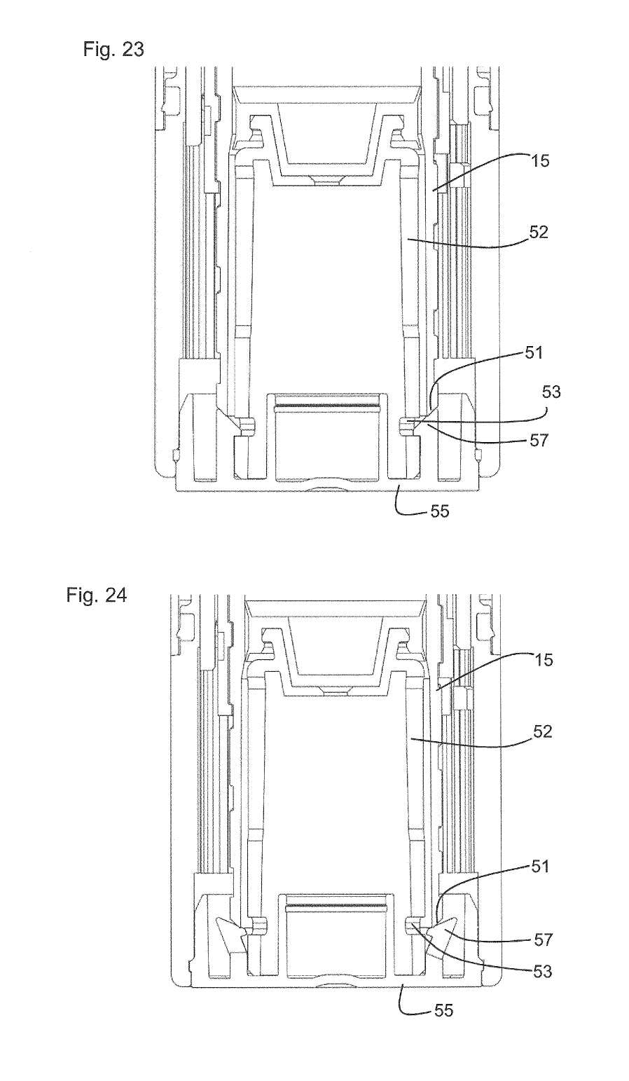

FIGS. 23 and 24 show a transportation lock of a bottom unit.

Like elements, elements of the same kind and identically acting elements may be provided with the same reference numerals in the figures.

In FIGS. 1A, 1B and 10, an inhalation device 1 is shown. The inhalation device 1 comprises a housing 3. The device 1 comprises an outer cylinder 4. The outer cylinder 4 is secured against axial movement with respect to the housing 3. The outer cylinder 4 is rotatable with respect to the housing 3. The inhalation device 1 further comprises a mouthpiece 6. The device 1 and the housing 3 have a distal end 41 and a proximal end 42. The term “distal end” designates that end of the device 1 or a component thereof which is or is to be arranged closest to the mouthpiece 6. The term “distal end” designates that end of the device 1 or a component thereof which is or is to be arranged closest to the dispensing end. The term “proximal end” designates that end of the device 1 or a component thereof which is or is to be arranged furthest away from the mouthpiece 6. The term “proximal end” designates that end of the device 1 or a component thereof which is or is to be arranged furthest away from the dispensing end. The distal end 41 and the proximal end 42 are spaced apart from one another in the direction of an axis 16. The axis 16 may be the main longitudinal axis or rotational axis of the device 1.

The inhalation device 1 comprises a detachable member 7. In particular, the detachable member 7 is a cap. The detachable member 7 is used for covering the mouthpiece 6. In FIGS. 1B and 10, the detachable member 7 is mounted onto the device 1 for covering the mouthpiece 6. In FIG. 1A, the detachable member 7 is removed from the device 1. The detachable member 7 may comprise a thread, preferably a screw thread. The detachable member 7 may be rotatable with respect to the housing 3 for screwing the detachable member 7 onto the device 1 and for unscrewing the detachable member 7 from the device 1. The outer cylinder 4 is rotationally fixed to the detachable member 7. In particular, the outer cylinder 4 follows rotation of the detachable member 7 with respect to the housing 3. For the detailed description of the components of the inhalation device 1 and their mechanical cooperation it is referred to document WO 2009/065707 A1.

The device 1 comprises a storage chamber 15. The storage chamber 15 holds one dose, preferably a plurality of doses, of a medical substance 2. The substance 2 may be a powder. In particular, the plurality of doses may correspond to a predefined number of doses, such that after the predefined number of doses has been delivered a lock-out mechanism (not explicitly shown in the Figures) may prevent a further operation of the device.

A numerical value corresponding to the predefined number of doses is a starting value of a counting mechanism. Before delivery of a first dose, the counting mechanism displays this predefined number as the number of available doses and with every dose delivery the number is decremented. Alternatively, the counting mechanism may display the number of doses that already have been delivered. In this case, the counting mechanism displays “0” as predefined number before delivery of the first dose and with every dose delivery the number is incremented. The counting mechanism is not shown in FIGS. 1A, 1B, 1C, but the dose counting mechanism will be discussed later on in detail.

The storage chamber 15 is terminated by a chamber sealing 24. The chamber sealing 24 is formed integrally with a top wall of the storage chamber 15. The device 1 further comprises a rotary part 25. The rotary part 25 is of substantially plate-like configuration and is connected in a rotationally fixed manner to the outer cylinder 4. Accordingly, the rotary part 25 follows rotation of the detachable member 7 and, hence, of the outer cylinder 4 about the main longitudinal axis or rotational axis 16 of the device 1 with respect to the storage chamber 15. However, the rotary part 25 is axially fixed relative to the housing 3.

The device 1 further comprises a metering rod 33. The metering rod 33 may be connected to the detachable member 7 by a snap fit element 34 when the detachable member 7 is attached to the housing 3. When the detachable member 7 is re-attached to the housing 3, the metering rod 33 travels axially in the proximal direction such that a most proximal part of the metering rod 33 comprising a metering chamber 40 enters the storage chamber 15. When the detachable member 7 is detached from the housing 3 the metering rod 33 travels axially in the distal direction such that the most proximal part of the metering rod 33 exits the storage chamber 15. The metering rod 33 is configured for functioning as a moving metering chamber 40 for a sub-quantity 14 of the substance 2 which is to be dispensed during a specific delivery action. The metering chamber 40 is provided in that end section of the metering rod 33 which projects into the substance 2, e.g. the proximal end section.

The inhalation device 1 further comprises a flow path comprising a flow channel 60 and an intermediate channel portion 61. The inhalation device 1 further comprises an actuation element 54. The actuation element 54 is arranged within an actuator housing 35. The actuator housing 35 is configured substantially as a hollow body. The actuator housing 35 is secured against axial movement with respect to the housing 3. The actuator housing 35 is adapted and arranged to guide an axial movement of the actuation element 54. The actuator housing 35 is arranged within the inner cylinder 4. The actuation element 54 and the actuator housing 35 are prevented from rotation with respect to one another.

The actuation element 54 comprises a piston comprising tongues 77 and a head 76. The actuation element 54 has a first and a second position. The first position is more proximal than the second position. In the first position, the tongues 77 of the actuation element 54 are configured to block the flow path between the flow channel 60 and the intermediate channel portion 61. In the second position, the actuation element 54 is positioned more distally, i.e. closer to the mouthpiece 6, such that the tongues 77 no longer block the flow path between the flow channel 60 and the intermediate channel portion 61.

The detachable member 7 is removed from the housing 3 by unscrewing the detachable member 7 from the housing 3. Accordingly, the detachable member 7 performs a concurrent axial movement in the distal direction and a rotational movement. The detachable member 7 and the rotary part 25 are in a splined engagement when the detachable member 7 is attached to the housing 3. During detachment of the detachable member 7 from the housing 3, the rotational movement of the detachable member 7 is transferred into a rotation of the rotary part 25 around the longitudinal axis 16 due to their splined engagement. The rotation of the rotary part 25 is transferred into a rotation of the actuation element 54. Furthermore, the concurrent axial and rotational movement of the detachable member 7 is transferred to the metering rod 33 concurrently performing an axial movement in the distal direction and a rotational movement around the longitudinal axis 16. As the detachable member 7 approaches the end of the threaded connection to the housing 3, the snap fit element 34 disengages from the metering rod 33. During detachment of the detachable member 7 from the housing 3, the actuation element 54 is not moved in an axial direction with respect to the housing 3. Accordingly, the actuation element 54 is in the first position before and after detachment of the detachable member 7 from the housing 3.

When the detachable member 7 is fully detached from the housing 3, the metering chamber 40 is in a first condition. The first condition of the metering chamber 40 is defined by the tongues 77 of the actuation element 54 closing the metering chamber 40 such that the metering chamber 40 is not in contact with the flow path. Accordingly, when the actuation element 54 is in the first position and the detachable member 7 is detached from the housing 3, the metering chamber 40 is in the first condition. In the first condition of the metering chamber 40, the tongues 77 of the actuation element 54 cover the metering chamber 40 on each side. Accordingly, in this first condition, it is not possible for the sub-quantity 14 of substance to trickle out. Rather, the substance 2 is reliably retained in the metering chamber 40.

After the detachable member 7 has been demounted or detached from the housing, the user may trigger an inhalation operation by subjecting the device to a suction airstream, in the simplest case by the user breathing in. Air is sucked in via the mouthpiece 6, and this, in first instance, by virtue of the head 76 being subjected to the action of air, results in the actuation element 54 being displaced axially towards the mouthpiece 6, i.e. in the distal direction. By virtue of the axially displaced actuation element 54, the tongues 77 are likewise displaced axially, in order to release the metering chamber 40. The metering chamber 40 is then in a second condition. The second condition of the metering chamber 40 is defined by the actuation element 54 being in the second position. In the second condition, the metering chamber 40 lies freely in the flow path between the flow channel 60 and the intermediate channel portion 61. The metering chamber 40 is cleared out with air being sucked from the flow channel 60.

After the inhalation operation was finished, the detachable member 7 can be re-attached to the housing 3. During attachment of the detachable member 7 to the housing 3, the detachable member 7 is moved axially in the proximal direction and concurrently rotated around the longitudinal axis 16. During this movement of the detachable member 7, the detachable member 7 abuts the metering rod 33 and pushes the metering rod 33 in the proximal direction. In an initial part of the attachment movement of the detachable member 7, the detachable member 7 does not engage with the metering rod 33 via the snap fit element 34, but only pushes the metering rod 33 in the proximal direction. Accordingly, if the user tried to move the detachable member in the distal direction during this initial part of the movement, the metering rod 33 would not follow such a movement of the detachable member 7 in the distal direction. Therefore, if the user only partly attached the detachable member 7 and then detached the detachable member 7, the user could not move the metering rod 33 in the distal direction in this way.

During the initial part of the attachment movement, the detachable member 7 passes a first position relative to the housing 3. In the first relative position, the detachable member 7 interacts with the counter drive member 19, thereby a counted number is caused to be incremented or decremented either directly or during subsequent detachment.

During a second part of the attachment movement of the detachable member 7 to the housing 3, the detachable member 7 engages with the metering rod 33 via the snap fit element 34. The second part of the movement follows after the initial part of the movement is finished. In particular, at the end of the initial part which corresponds to the beginning of the second part, the metering rod 33 has been moved in the proximal direction so far that it abuts an end of travel face preventing a further pushing of the metering rod 33 in the proximal direction. Further, at the beginning of the second part of the attachment movement, the detachable member 7 is in a second position relative to the housing 3. This second position cannot be reached without having passed the first relative position previously. In the second part of the attachment movement, the detachable member 7 continues to travel in the proximal direction. This causes the engagement of the snap fit element 34 with the metering rod 33.

Accordingly, the metering rod 33 is moved into the proximal direction when the detachable member 7 is attached to the housing 3. Further, the metering rod 33 follows a rotation of the detachable member 7 in the second part of the attachment movement. During re-attachment of the detachable member 7 to the housing 3, the metering rod 33 is moved in the proximal direction due to the interaction with the detachable member 7. Movement of the metering rod 33 in the proximal direction is transferred to the actuation element 54 (if the actuation element 54 is in the second position). Thereby, the actuation element 54 is moved from the second position to the first position. However, in case that the detachable member 7 is detached from the housing 3 and, afterwards, re-attached to the housing 3 without a drug delivery being performed in the meantime, the actuation element 54 remains in the first position the whole time. Accordingly, as the actuation element 54 is already in the first position, it cannot be moved axially during attachment of the detachable member 7 to the housing 3.

FIG. 2 shows a perspective view of a counting member 5. The counting member 5 has a tubular shape. The counting member 5 defines a longitudinal axis which is parallel to the rotational axis 16 of the inhalation device 1. The counting member 5 comprises an outer surface 8 and an inner surface 9. The counting member 5 comprises indicia 10. The indicia 10 are arranged on the outer surface 8 of the counting member 5. The counting member 5 is configured to be arranged inside the housing 3 such that at least one of the indicia 10 is visible through a window 89 of the housing 3. The indicia 10 are numbers corresponding to the number of doses left in the device 1 or to the numbers of dispensed doses. Alternatively or additionally, the indicia 10 may be color-coded. For example, the indicia 10 may be coloured such that, when only a low number of doses is left in the device, e.g. five doses, the colour of the indicia 10 visible in the window may change, warning the user that the device 1 will have to be replaced soon.

Moreover, the counting member 5 comprises gear teeth 11 which are arranged on the outer surface 8. The gear teeth 11 are arranged along a helical path. As will be described in detail later on, the gear teeth 11 are configured to interact with an engagement feature of a counter drive member. In particular, the gear teeth 11 are shaped such that the counting member 5 follows a rotation of the counter drive member in a second rotational direction.

Further, the counting member 5 comprises a claw 12. The claw 12 protrudes outwardly from the outer surface 8 of the counting member 5. The claw 12 is arranged at a proximal end of the counting member 5. The claw 12 has a first surface 13 and a second surface 17. The first surface 13 of the claw 12 is roughly perpendicular to an azimuthal direction around the rotational axis of the counting member 5. The first surface 13 of the claw 12 extends roughly in a radially outward direction away from the rotational axis of the counting member 5. The second surface 17 of the claw 12 forms a flatter angle to the azimuthal direction than the first surface 13.

As will be described later in detail, the claw 12 is configured to interact with axially extending ribs arranged at an inner surface of the housing 3. The first surface 13 of the claw 12 are shaped such that the counting member 5 is prevented from rotating in a first rotational direction relative to the housing 3 due to the engagement of the claw 12 and the axially extending ribs. Further, the second surface 17 of the claw 12 is shaped such that the counting member 5 is enabled to rotate in the second rotational direction relative to the housing 3 as the second surface 17 of the claw 12 is enabled to slide over a correspondingly tapered surface of the axially extending ribs in this case.

Further, the counting member 5 comprises a slide rail 18. The slide rail 18 is arranged at the inner surface 9 of the counting member 5. The slide rail 18 has a helical shape. The slide rail 18 of the counting member 5 is configured to be engaged with a corresponding slide rail of the storage chamber 15. Thereby, the counting member 5 is enabled to travel along a helical path relative to the storage chamber 15 and relative to the housing 3 as the storage chamber 15 is fixed to the housing 3. The position of the counting member 5 relative to the housing 3 along this path corresponds to the current value of the counted number. This position also determines which one of the indicia 10 is visible in the window 89 of the housing 3.

Moreover, a first abutment surface is provided on the counting member 5 which is not visible in FIG. 2. The first abutment surface is arranged at the outer surface 8 of the counting member 5. The first abutment surface is arranged in the vicinity of the distal end of the counting member 5. The first abutment surface is configured to be abutted by a second abutment surface provided on a counter drive member, thereby preventing a further rotation of the counting member 5 relative to the counter drive member. The first abutment surface faces in the azimuthal direction.

FIG. 3 shows a perspective view of a counter drive member 19. The counter drive member 19 is an essentially ring-shaped element. The counter drive member 19 comprises a slot 20 which extends in an axial direction. Thus, the ring-shape of the counter drive member 19 is not closed, thereby forming a C-shape. As will be discussed later on, the slot 20 facilitates the manufacturing process.

Further, a second abutment surface 21 is provided on the counter drive member 19. In particular, the counter drive member 19 comprises a lug 22 protruding in the distal direction wherein the second abutment surface 21 is provided on the lug 22. The second abutment surface 21 faces in the azimuthal direction around a rotational axis of the counter drive member 19. The rotational axis of the counter drive member 19 is parallel to the rotational axis of the inhalation device 1. As will be discussed later, the second abutment surface 21 is configured to abut the first abutment surface of the counting member 5, thereby preventing a further rotation of the counter drive member 19 relative to the counting member 5.

Further, the counter drive member 19 comprises a spring element 23. When the counter drive member 19 is assembled to the inhalation device 1, the spring element 23 is configured to bias the counter drive member 19 in the axial direction, in particular in the proximal direction. Thereby, the spring element 23 ensures that the counter drive member 19 remains in its second position when the detachable member 7 is detached from the housing 3.

In particular, the spring element 23 comprises four spring arms wherein a first pair of two spring arms form a V-shape and a second pair of two spring arms is arranged opposite to the first pair of spring arms and also forms a V-shape. This design further stabilizes the counter drive member 19. In an alternative design, which is not shown in FIG. 3, the spring element 23 comprises two spring arms which are arranged opposite to each other.

The counter drive member 19 comprises an inner surface 26 and an outer surface 27. An engagement feature 28 is arranged at the inner surface 26 of the counter drive member 19. The engagement feature 28 is a tooth. The engagement feature 28 of the counter drive member 19 is configured to interact with the gear teeth 11 of the counting member 5. The engagement feature 28 is shaped such that the counter drive member 19 is enabled to rotate in the first rotational direction relative to the counting member 5. In this case, the engagement feature 28 slides over a side surface of the respective tooth and engages with the next tooth. Further, the engagement feature 28 is shaped such that the counter drive member 19 is prevented from rotating in the second rotational direction relative to the counting member 5 wherein the second rotational direction is opposite to the first rotational direction.

Further, the counter drive member 19 comprises a protrusion 29. The protrusion 29 is arranged on the outer surface 27 of the counter drive member 19. The protrusion 29 protrudes in the radially outward direction. The protrusion 29 has a first side surface 30 and a second side surface 31 which are spaced apart from each other in the azimuthal direction. The azimuthal direction circles around the rotational axis of the counter drive member 19.

Further, the protrusion 29 comprises a third surface 32 which connects the first and the second side surface 30, 31. The third surface 32 extends essentially in the azimuthal direction. The third surface 32 is inclined relative to the azimuthal direction. Moreover, the protrusion 29 also comprises a fourth surface 36 which is parallel to the third surface 32 and also connects the first and the second side surface 30, 31.

The third surface 32 is configured to slide along a cut-out in the housing 3 when the counter drive member 19 travels along a helical path relative to the housing 3. Due to the inclination of the third surface 32, the friction of the counter drive member 19 and the housing 3 can be kept to a minimum. The first and the second surface 30, 31 are configured to abut a respective end face of the cut-out, thereby limiting the range of motion of the counter drive member 19.

In particular, the counter drive member 19 may be constrained such that it can only be moved from a first to a second position and vice versa. The counter drive member 19 is in its first position when the detachable member 7 is fully attached to the housing 3. The counter drive member 19 is moved into its first position during the incrementing or decrementing the counted number. The counter drive member 19 is in its second position when the detachable member 7 is partially or fully detached from the housing 3. The second position of the counter drive member 19 is position wherein the counter drive member 19 is ready to increment or decrement a counted number.

The protrusion 29 of the counter drive member 19 is configured to be engaged by a first and a second projection of the detachable member 7. Thereby, the protrusion 29 and the respective projection are configured to transfer a movement of the detachable member 7 into a movement of the counter drive member 19. In particular, the counter drive member 19 is configured to follow a rotation of the detachable member 7 when the projection 29 is engaged with one of the protrusions.

The counter drive member 19 further comprises an identical second protrusion 29′ which is arranged opposite to the first protrusion 29 and which is not visible in the perspective view shown in FIG. 3. As the function of the identical second protrusion 29′ is essentially identical to the protrusion 29, only the protrusion 29 is referred to in the following.

FIG. 4 shows a cross-sectional view of the detachable member 7. The detachable member 7 comprises a thread 37 arranged at its inner surface. The detachable member 7 can be detached, e.g. unscrewed, from the housing 3 of the inhalation device 1. The thread 37 of the detachable member 7 is engaged with a thread on the housing 3 when the detachable member 7 is attached to the housing 3.

The detachable member 7 comprises axially extending splines 38. When the detachable member 7 is attached to the housing 3, the detachable member 7 and the rotary part 25 are in a splined engagement due to an engagement of the splines 38 of the detachable member 7 with corresponding elements of the rotary part 25.

The detachable member 7 comprises the first projection 39 and the second projection 43. The first projection 39 and the second projection 43 are arranged at the inner surface of the detachable member 7. The first and the second projection 39, 43 protrude inwardly from the inner surface. The first and the second projection 39, 43 are spaced apart in the azimuthal direction and in the axial direction. In particular, the first projection 39 is arranged more distally than the second projection 43. Moreover, the detachable member 7 comprises a second pair of a first projection 39′ and a second projection 43′ which are identical to the previously described first and second projection 39, 43. The second pair of projections 39′, 43′ is arranged opposite to the first and the second projection 39, 43.

The first and the second projection 39, 43 of the detachable member 7 are configured to engage with the protrusion 29 of the counter drive member 19. Further, the second pair of projections 39′, 43′ is configured to engage with the second protrusion 29′ of the counter drive member 19. As the second pair of projections 39′, 43′ is substantially identical to the first and the second projection 39, 43, only the first and the second projection 39, 43 are described in the following.

In particular, the detachable member 7 is configured such that the detachable member 7 is rotationally locked to the counter drive member 19 when one of the first or the second projection 39, 43 is engaged with the protrusion 29 of the counter drive member 19. In other words, the counter drive member 19 follows a rotation of the detachable member 7 when one of the first or the second projection 39, 43 is engaged with the protrusion 29 of the counter drive member 19.

The detachable member 7 is further configured to seal the inhalation device 1 against moisture when the detachable member 7 is completely attached to the housing 3.

FIG. 5A and FIG. 5B show perspective views of the housing 3. FIG. 5C shows an enlarged view of a part of the housing 3.

The housing 3 comprises a thread 44. The thread is arranged at the distal part of the housing 3. The detachable member 7 can be screwed along the thread 44.

Further, the housing 3 comprises the window 89. A part of the counting member 5 is visible through the window 89. In particular, the window 89 is configured such that one indicia 10 at a time is visible through the window 89. The window 89 is arranged such that the window 89 is visible when the detachable member 7 is attached to the housing 3 and when the detachable member 7 is detached from the housing 3.

Further, the housing 3 comprises a cut-out 45. The cut-out 45 is arranged such that it is inclined relative to an azimuthal direction which circles around a longitudinal axis defined by the housing 3. The longitudinal axis of the housing 3 is parallel to the rotational axis 16 of the inhalation device 1.

The inclination of the cut-out 45 corresponds to the inclination of the protrusion 29 of the counter drive member 19. The counter drive member 19 is configured to be arranged at least partially inside the housing 3. Further, when the counter drive member 19 is arranged inside the housing 3, the protrusion 29 protrudes out of the cut-out 45 of the housing 3.

Further, as can be seen in FIG. 5B, an orientation segment 46 is arranged at a distal end of the housing 3 protruding inwardly. The orientation segment 46 ensures that the storage chamber 15 can be fixed to the housing 3 only in one orientation.

Further, the housing 3 comprises axially extending ribs 47. The axially extending ribs 47 are arranged at the inner surface of the housing 3. The axially extending ribs 47 are configured to cooperate with the claw 12 of the counting member 5. In particular the claw 12 and the axially extending ribs 47 are configured to allow a rotation of the counting member 5 relative to the housing 3 in the second rotational direction and to prevent a rotation of the counting member 5 relative to the housing 3 in the first rotational direction.

FIG. 6 shows a perspective view of the storage chamber 15. The storage chamber 15 is configured to be fixed relative to the housing 3.

Moreover, the storage chamber 15 comprises a slide rail 48. The slide rail 48 has a helical shape. The slide rail 48 is arranged at an outer surface of the storage chamber 15. The slide rail 48 of the storage chamber 15 is configured to engage with the slide rail 18 arranged at the inner surface of the counting member 5. Accordingly, the counting member 5 can be screwed up and down the storage chamber 15. The counting member 5 is constrained such that it can only carry out a helical movement relative to the storage chamber 15 and, as the storage chamber 15 is fixed to the housing 3, the counting member 5 is constrained such that it can only carry out a helical movement relative to the housing 3.

The storage chamber 15 comprises a cut-out segment 49 at its distal end which is configured to engage with the orientation segment 46 of the housing 3, thereby ensuring that the storage chamber 15 is correctly aligned with the housing 3.

Further, the storage chamber 15 comprises a centering device 50 which is arranged at a proximal end of the storage chamber 15. In particular, a first and a second centering device 50 are arranged at opposite surface sides. The centering devices 50 align the storage chamber 15 inside the housing 3.

Further, a chamfered surface 51 is provided at the proximal end of the storage chamber 15. As will discussed later on, the chamfered surface 51 is configured to engage the storage chamber 15 with a snapper of a bottom lid. The snapper and the chamfered surface 51 form a transportation lock mechanism which ensures that a spring is allowed to release its tension only when a bottom unit comprising the spring, a spring housing, a spring housing seal and a bottom lid are attached to the remainder of the inhalation device 1.

Further, FIG. 7 shows a perspective view of the spring housing 52 which is configured to be arranged inside the storage chamber 15. The spring can be arranged inside the spring housing 52. The spring housing 52 comprises an opening 53. When the transportation lock mechanism is activated, the spring housing is fixed to the bottom lid.

FIG. 8 shows a perspective view of the bottom lid 55. The bottom lid 55 comprises a stabilizing element 56 which is configured to stabilize the spring housing 52 when the spring housing 52 is attached to the bottom lid 55. Further, the bottom lid 55 comprises a snapper 57.

FIG. 9 shows an exploded view of the inhalation device 1.

FIGS. 10 and 11 indicate the assembly process of the inhalation device 1. In one step, the counter drive member 19 is entered into the housing 3. Thereby, a tool is used to press the counter drive member 19 such that the slot 20 is narrowed. Thereby, the radius of the counter drive member 19 is slightly reduced. This allows entering the counter drive member 19 into the housing 3. Once the counter drive member 19 has reached its predetermined position, the tool is separated from the counter drive member 19, thereby allowing the counter drive member 19 to release its tension and to expand such that the width of the slot 20 increases. Now, the counter drive member 19 is fixed to the housing 3 as the protrusion 29 protrudes through the cut-out 45 of the housing 3. The counter drive member 19 is now constrained to move between its first position and its second position relative to the housing 3. FIG. 10 shows a stage before the counter drive member 19 is entered into the housing 3 and FIG. 11 shows a later stage of the assembly process wherein the counter drive member 19 has been entered into the housing 3.

Further, the counting member 5 is screwed onto the storage chamber 15 during the assembly process. The initial value of the counted number depends on the axial position of the counting member 15 relative to the storage chamber 15 at the end of the assembly process. As the storage chamber 15 is fixed to the housing 3, the initial value of a counted number is determined by the axial position of the counting member 5 relative to the housing 3 at the end of the assembly process. This position determines how many counting operations can be carried out before the first abutment surface and the second abutment surface 21 abut each other.

The counted number corresponds to the indicia 10 visible through the window 89 of the housing 3. A large range of counted numbers is printed on the counting member 5, e.g. a range from “120” to “0”. However, the number of operations N is determined by the position of the counting member 5 relative to the housing 3. Thus, the same assembly can be used wherein the predetermined number N is set to different values during the assembly process by positioning the counting member 5 differently relative to the storage chamber 15 and relative to the housing 3. For example, in one embodiment, the counting member 5 is positioned such that the initial value of the counted number is a first value, e.g. “60”, and in another embodiment, the counting member 5 is positioned such that the initial value of the counted number is a different second value, e.g. “120”. FIG. 10 shows a stage before the counting member 5 is screwed onto the storage chamber 15 and FIG. 11 shows a later stage of the assembly process wherein the counting member 5 has been screwed onto the storage chamber 15.

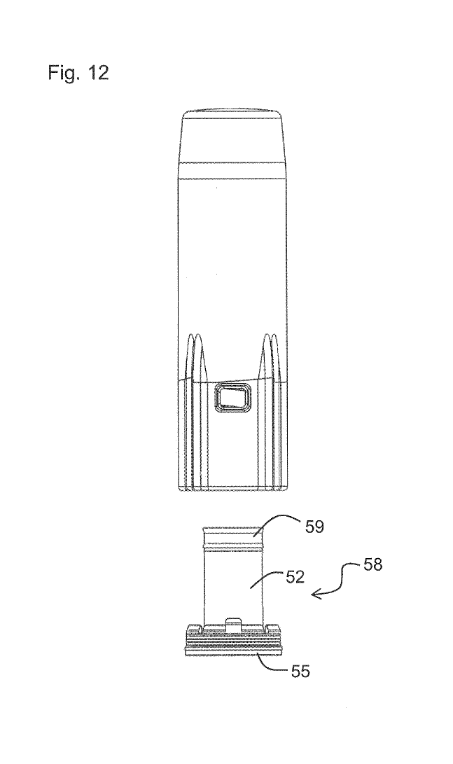

FIG. 12 shows a last step of the assembly process wherein the bottom unit 58 comprising the bottom lid 55, the spring housing 52, the spring (not visible) and a spring housing seal 59 is entered into the remained of the inhalation device 1.

To perform a drug dispense operation, the detachable member 7 has to be detached from the housing 3. Thereby, the mouthpiece 6 is uncovered. Moreover, the detachable member 7 is connected with the metering rod 33. The detachment of the detachable member 7 results in the metering rod 33 traveling axially in the distal direction such that a dose of the substance 2 is prepared for inhalation.

FIGS. 13 to 17 show the process of detaching the detachable member 7 from the housing 3. FIG. 13 shows an enlarged view of a part of the inhalation device 1 in a state wherein the detachable member 7 is completely attached to the housing 3. In the configuration shown in FIG. 13, the counter drive member 19 is in its first position. The protrusion 29 of the counter drive member 19 is engaged with the first projection 39 of the detachable member 7. In particular, the protrusion 29 of the counter drive member 19 abuts the first projection 39 of the detachable member 7.

To detach the detachable member 7 from the housing 3 the detachable member 7 has to carry out a detachment movement. The detachment movement is a rotation of the detachable member 7 relative to the housing 3 in the first rotational direction. The detachment movement comprises a first part and a second part. The first part is an initial part of the movement. The second part is a final part of the movement.

In the first part of the detachment movement, the detachable member 7 is rotated in the first rotational direction and the protrusion 29 remains engaged with the first projection 39. Thus, the counter drive member 19 follows the rotation of the detachable member 7 in the first rotational direction. The counter drive member 19 and the detachable member 7 are configured such that they are moved in different axial directions when rotated in the same rotational direction. This is due to the fact that the threads 37, 44 of the detachable member 7 and the housing 3 are oppositely orientated to the inclination of the cut-out 45 in the housing 3 and the inclination of the protrusion 29 of the counter drive member 19.

Accordingly, a can be seen in FIG. 14 which shows a configuration at the end of the first part of the detachment movement, the detachable member 7 travels in the distal direction. At the same time, the counter drive member 19 travels from its first position to its second position, i.e. in the proximal direction. Thereby, at the end of the first part of the detachment movement, the protrusion 29 disengages from the first projection 39.

In a second part of the detachment movement, the detachable member 7 continues to rotate in the first rotational direction relative to the housing 3. At the end of the second part, the detachable member 7 is completely unscrewed from the housing 3 and detached from the housing 3.

The protrusion 29 is not engaged with the first projection 39 of the detachable member 7 in the second part of the detachment movement. Thus, the counter drive member 19 does not follow the rotation of the detachable member 7 in the second part of the detachment movement. The counter drive member 19 remains in its second position.

FIGS. 15 and 16 show details of the interaction of the counter drive member 19 and the counting member 5 during the detachment of the detachable member 7. FIG. 15 corresponds to the configuration shown in FIG. 13 when the detachable member 7 is completely attached to the housing 3. FIG. 16 corresponds to the configuration shown in FIG. 14 at the end of the first part of the detachment movement.

In FIG. 15, the engagement feature 28 of the counter drive member 19 is engaged with one of the teeth of the gear teeth 11. During a rotational movement of the counter drive member 19, the engagement feature 28 slides over a ramp-shaped side surface of the tooth and snaps into the next tooth of the gear teeth 11. Each tooth of the gear teeth 11 comprises a ramp-shaped first side surface, thereby ensuring that the counter drive member 19 is enabled to rotate relative to the counting member 5 in the first rotational direction. Accordingly, the counter drive member 19 is rotated in the first rotational direction relative to the counting member 5 during detachment of the detachable member 7.

FIG. 17 shows details of the interaction of the counting member 5 and the housing 3. In particular, it can be seen that the claw 12 of the counting member 5 abuts one of the axially extending ribs 47 of the housing 3, thereby preventing a rotation of the counting member 5 in the first rotational direction relative to the housing 3. This ensures that the counting member 5 is not moved relative to the housing 3 during a detachment of the detachable member 7.

In particular, the first surface 13 of the claw 12 abuts a first surface 62 of the axially extending rib 47 wherein the first surface 13 of the claw 12 and the first surface 62 of the axially extending rib 47 are parallel to each other, thereby preventing the claw 12 from sliding over the axially extending rib 47.

It can also be seen in FIG. 17 that the second surface 17 of the claw 12 is enabled to slide over a tapered second surface 63 of the axially extending rib 47 such that a rotation of the counting member 5 relative to the housing 3 in the second rotational direction is enabled.

Once the detachable member 7 has been detached completely from the housing 3, the mouthpiece 6 is free from the detachable member 7 and the user can perform an inhalation.

After the inhalation has been performed, the detachable member 7 has to be reattached to the housing 3 and, afterwards, be detached from the housing 3 again before a subsequent inhalation can be performed. To reattach the detachable member 7 to the housing 3, the detachable member 7 can be screwed onto the housing 3 along the thread 44 of the housing 3. Thereby the detachable member 7 carries out an attachment movement. The attachment movement comprises a first part and a subsequent second part.

During the first part of the attachment movement, the detachable member 7 is not attached to the counter drive member 19 such that the detachable member 7 carries out a helical movement with a rotational component in the second rotational direction relative to the counter drive member 19 and relative to the housing 3. The counter drive member 19 remains in its second position during the first part of the attachment movement.

FIG. 18 shows a detailed view of a part of the inhalation device 1 at the end of the first part of the attachment movement. At the end of the first part of the attachment movement, the second projection 43 abuts the protrusion 29 of the counter drive member 19, thereby engaging the detachable member 7 and the counter drive member 19. In the second part of the attachment movement, the detachable member 7 is rotated further in the second rotational direction. Due to the engagement of the second projection 43 and the protrusion 29, the counter drive member 19 follows the rotation of the detachable member 7 in the second rotational direction. As the counter drive member 19 is constrained to carry out a helical movement, it is moved from its second position back to its first position. Accordingly, the counter drive member 19 is moved in the distal direction. The detachable member 7 travels in the proximal direction during the attachment movement.

FIG. 19 shows the assembly at the end of the attachment process. The counter drive member 19 has reached its first position and the detachable member 7 is fully reattached to the housing.

FIG. 20 shows the interaction of the counter drive member 19 and the counting member 5 during the attachment of the attachable member 7 to the housing 3. The engagement feature 28 of the counter drive member 19 and the gear teeth 11 of the counting member 5 are configured such that the counting member 5 follows a rotation of the counter drive member 19 in the second rotational direction. Accordingly, the counting member 5 is rotated in the second rotational direction when the detachable member 7 is attached to the housing 3. Thereby, a counted number counted by the counting member 5 is increased or decreased. In particular, as the counting member 5 is rotated, another indicia 10 is now visible through the window 89 of the housing 3.

Accordingly, the counted number is incremented or decremented each time the detachable member 7 is attached to the housing 3. During normal use of the inhalation device 1, this will correspond to a drug dispense operation having been carried out.

The mechanism is configured such that the incrementing or decrementing of the counted number will stop shortly before the snap fit element 34 snaps into the metering rod 33 such that it is impossible to carry out a drug dispense operation without incrementing or decrementing the counted number.

Furthermore, the inhalation device 1 is configured such that no drug dispense operation can be performed after N operations have been carried out, wherein N is a predefined number which is determined by the initial relative position of the counting member 5 relative to the housing 3. This lock-out mechanism is described in detail with reference to FIGS. 21 and 22.

The lock-out mechanism prevents the user from trying to perform a drug dispense operation when the device is empty. Accordingly, it increases the safety and usability. In particular, the assembly is designed such that the detachable member is prevented from being detached from the housing after it has been detached N times. Thus, it is ensured that a maximal number of N drug delivery operations can be carried out as only one drug delivery operation can be carried out after the detachable member is detached before it is reattachted to the housing.

FIG. 21 shows a perspective view of the interaction of the counter drive member 19 and the counting member 5 when the detachable member 7 has been detached N times. FIG. 22 shows the interaction of the counter drive member 19 and the detachable member 7 in a configuration wherein the detachable member 7 has been detached N times and, afterwards, has been reattached to the housing 3.

In particular, the assembly is configured such that, after detachment of the detachable member 7 from the housing 3 for an Nth time, the detachable member 7 is enabled to be reattached to the housing 3 and, afterwards, the detachable member 7 is prevented from being detached from the housing 3 again. In particular, the detachable member 7 being prevented from being detached from the housing 3 again is to be understood such that the detachable member 7 can only be detached from the housing 3 by damaging the inhalation device 1.

The first abutment surface 64 is provided on the counting member 5. The second abutment surface 21 is provided on the counter drive member 19. Every time the counted number is incremented or decremented, the first and the second abutment surface 64, 21 are moved closer together along a helical path. After the detachable member 7 is detached from the housing 3 an Nth time, the first and the second abutment surface 64, 21 abut each other. It should be noted that the detachable member 7 is detached from housing 3 in this state and, therefore, the detachable member 7 is also disengaged from the metering rod 33. FIG. 21 shows the abutment of the first and the second abutment surface 64, 21. Thereby, the first and the second abutment surface 64, 21 prevent that the counter drive member 19 is rotated relative to the counting member 5 in the first rotational direction. Moreover, as the counter drive member 19 is in its first position, it cannot be rotated further in the second rotational direction.

Then, the detachable member 7 is re-attached to the housing 3. The assembly is thereby moved into the configuration shown in FIG. 22. As discussed above, the detachable member 7 engages the metering rod 33 via the snap fit element 34 during this movement when the detachable member reaches the second position relative to the housing 3. The assembly is configured such that the second relative position can only be reached during the reattachment of the detachable member 7 to the housing 3 after the Nth detachment when the assembly is in a configuration wherein it is no longer possible to detach the detachable member 7 from the housing 3. In particular, the second position is reached only after an abutment of second projection 43 of the detachable member 7 with the the protrusion 29 of the counter drive member 19 has happened.

The detachable member 7 cannot be detached from the housing 3 anymore as the counter drive member 19 is prevented from being moved out of its first position. To move the counter drive member 19 out of its first position, the counter drive member 19 has to be rotated in the first rotational direction relative to the housing 3 which is prevented by the abutment of the first and the second abutment surface 64, 21. Further, the detachable member 7 is prevented from rotating in the first rotational direction as it is rotationally locked to the counter drive member 19.

However, the detachable member 7 can be rotated by a small angle until the first projection 39 abuts the protrusion 29 of the counter drive member 19 as shown in FIG. 22. The small angle is smaller than 10°, preferably smaller than 5°. The metering rod 33 which is engaged with the detachable member 7 via the snap fit element 34 in this state cannot be moved in the distal direction far enough to remove a sub-quantity 14 of the drug from the storage chamber 15 as the detachable member 7 can only be rotated by the small angle. The second relative position at which the detachable member 7 engages with the metering rod 33 lies within the range by which the detachable member 7 is allowed to be rotated.

As the metering rod 33 engages with the detachable member 7 via the snap fit element 34 only after the first position has been passed, it is prevented that the user only partly reattaches the detachable member 7 and afterwards detaches the partly detachable member 7 and, thereby, tries to move a sub-quantity of the dose out of the dosing chamber without incrementing or decrementing the counted number. This is not possible as the counted number is incremented or decremented before an engagement of the detachable member 7 with the metering rod 33 which results in the metering rod 33 following a movement of the detachable member 7 in the distal direction.

This configuration corresponds to an orientation of the counting member 5 wherein the indicia “0” is visible through the window 89, thereby indicating to a user that the device 1 is empty. As the detachable member 7 cannot be detached from the housing 3 anymore, the user is warned that the device 1 is empty. The fact that the detachable member 7 is allowed to rotate by a small angle before being locked makes it obvious for the user that the detachment is prevented on purpose.

FIGS. 23 and 24 show the transportation lock of the bottom unit 58. Before the assembly of the bottom unit 58 to the remainder of the inhalation device, the snapper 57 based in the bottom lid 55 are engaged with the opening 53 in the spring housing 52, thereby preventing release of the tension of the spring, as shown in FIG. 23.

When the bottom unit 58 is attached to the remainder of the inhalation device 1, as shown in FIG. 24, the chamfered surface 51 of the storage chamber 15 engages the snapper 57 and pushes the snapper 57 in the radial outward direction. Thereby, the snapper 57 is disengaged from the spring housing 52. Now, the spring is free to release its tension, thereby moving the powder reservoir in the distal direction.

### REFERENCE NUMERALS

1 inhalation device

2 substance

3 housing

4 outer cylinder

5 counting member

6 mouthpiece

7 detachable member

8 outer surface of the counting member

9 inner surface of the counting member

10 indicia

11 gear teeth

12 claw

13 first surface of the claw

14 sub-quantity of substance

15 storage chamber

16 rotational axis

17 second surface of the claw

18 slide rail of the counting member

19 counter drive member

20 slot

21 second abutment surface

22 lug

23 spring element

24 chamber sealing

25 rotary part

26 inner surface of the counter drive member

27 outer surface of the counter drive member

28 engagement feature

29 protrusion

30 first side surface of the protrusion

31 second side surface of the protrusion

32 third surface of the protrusion

33 metering rod

34 snap fit element

35 actuator housing

36 fourth surface of the protrusion

37 thread of the detachable member

38 spline

39 first projection

40 metering chamber

41 distal end

42 proximal end

43 second projection

44 thread of the housing

45 cut-out

46 orientation segment

47 axially extending rib

48 slide rail of storage chamber

49 cut-out segment

50 centering device

51 chamfered surface

52 spring housing

53 opening of the spring housing

54 actuating element

55 bottom lid

56 stabilizing element

57 snapper

58 bottom unit

59 spring housing seal

60 flow channel

61 intermediate channel portion

62 first surface of the axially extending rib

63 second surface of the axially extending rib

64 first abutment surface

76 head

77 tongues

89 window

1-30. (cancelled)

31. An assembly for a dry powder inhaler comprising
a housing,
a detachable member which is detachable from the housing and attachable to the housing, and
a mechanism configured to be transferred from one state to a subsequent state when the detachable member is detached from the housing.

32. The assembly according to claim 31 further comprising
a first abutment surface, and
a second abutment surface,
wherein the assembly is configured such that, during detachment of the detachable member from the housing for an Nth time, N being a predetermined number, the first abutment surface is brought into abutment with the second abutment surface,
wherein the assembly is configured such that, when the first abutment surface abuts with the second abutment surface, the detachable member is enabled to be reattached to the housing and, afterwards, the detachable member is prevented from being detached from the housing.

33. The assembly according to claim 32,
wherein the detachable member is allowed to be rotated by a small angle relative to the housing when the detachable member is prevented from being detached from the housing.

34. The assembly according to claim 32,
wherein the first abutment surface is provided on a counting member comprising indicia,
wherein the second abutment surface is provided on a counter drive member,
wherein the counting member is configured to follow a rotation of the counter drive member in the second rotational direction relative to the housing and wherein the counting member is prevented from following a rotation of the counter drive member in the first rotational direction relative to the housing.

35. The assembly according to claim 34,
wherein the predetermined number N is determined by the position of the counting member relative to the housing.

36. The assembly according to claim 34,
wherein the counter drive member and the counting member are configured such that, when the first abutment surface abuts the second abutment surface, the counter drive member is prevented from rotating relative to the counting member.

37. The assembly according to claim 34,
wherein the assembly is configured such that, when the counter drive member is prevented from rotating relative to the counting member in the first rotational direction, the detachable member is prevented from rotation in the first rotational direction relative to the housing, thereby also being prevented from being detached from the housing.

38. The assembly according to claim,31
wherein the dry powder inhaler is configured such that a drug dispense operation is permitted only when the detachable member is detached from the housing.

39. The assembly according to claim 31,
wherein the dry powder inhaler is configured such that, after a drug dispense operation has been carried out, the detachable member has to be reattached to the housing and, afterwards, the detachable member has to be detached from the housing to enable a subsequent drug dispense operation.

40. The assembly according to claim 31,
wherein the detachable member is configured to cover a mouthpiece of the dry powder inhaler when the detachable member is attached to the housing.

41. The assembly according to claim 31,
wherein the detachable member is configured such that, during detachment of the detachable member from the housing, the detachable member is rotated in a first rotational direction relative to the housing,
wherein the detachable member is configured such that, during attachment of the detachable member to the housing, the detachable member is rotated in a second rotational direction relative to the housing which is opposite to the first rotational direction.

42. The assembly according to claim 31,
wherein the detachable member is configured to engage with a metering rod during attachment of the detachable member to the housing, and
wherein the assembly is configured such that, during the attachment of the detachable member to the housing, the detachable member engages with the metering rod in a second relative position to said housing, and wherein said second relative position not being reachable during attachment before having passed a first relative position where a counted number is caused to be incremented or decremented either directly or during subsequent detachment.

43. The assembly according to claim 42,
wherein the assembly is configured such that during the attachment of the detachable member to the housing after the detachable member has been detached N times, the second relative position is not reachable during attachment before
the detachable member has reached a configuration wherein it is no longer possible to detach the detachable member from the housing.

44. The assembly according to claim 42,
wherein the detachable member is allowed to be rotated by a small angle relative to the housing when the detachable member is prevented from being detached from the housing, and
wherein the second position of the detachable member relative to the housing is within the small angle relative to the housing by which the detachable member is allowed to be rotated when the detachable member is prevented from being detached from the housing.

45. The assembly according to one of claim 42,
wherein the detachable member is configured to engage with the metering rod in such a manner that the metering rod removes a sub-quantity of a drug from a storage chamber during detachment of the detachable member from the housing, thereby setting the sub-quantity as a dose for the next drug dispense operation.

46. The assembly according to one of claim 42,
wherein the metering rod is configured to travel in the proximal direction during attachment of the detachable member to the housing,
wherein the metering rod is in a most proximal position when the detachable member is fully attached to the housing,
wherein the detachable member is configured to engage with the metering rod in a manner such that the metering rod follows a distal movement of the detachable member only when the metering rod is in its most proximal position.

47. The assembly according to claim 31,
comprising a counter drive member configured to increase or decrease a counted number when the counter drive member is rotated in a second rotational direction relative to the housing, and
wherein the detachable member is configured such that, during detachment of the detachable member from the housing, the detachable member is rotated in a first rotational direction relative to the housing and the counter drive member follows the rotation of the detachable member in the first rotational direction, and
wherein the detachable member is configured such that, during attachment of the detachable member to the housing, the detachable member is rotated in the second rotational direction relative to the housing which is opposite to the first rotational direction and the counter drive member follows the rotation of the detachable member in the second rotational direction.

48. The assembly according to claim 47,
wherein the counter drive member is constrained such that it can only carry out a helical movement relative to the housing,
wherein the detachable member is constrained such that it can only carry out a helical movement relative to the housing, and
wherein the counter drive member and the detachable member are configured such that, when the counter drive member follows a rotational movement of the detachable member, the counter drive member carries out a helical movement with an axial component being directed opposite to an axial component of the helical movement carried out by the detachable member.

49. The assembly according to claim 47,
wherein the counter drive member comprises a protrusion,
wherein the detachable member comprising a first projection and a second projection, and
wherein the detachable member is configured such that, when the detachable member rotates in the first rotational direction, the first projection engages the protrusion of the counter drive member such the counter drive member follows the rotation of the detachable member in the first rotational direction, and
wherein the detachable member is configured such that, when the detachable member rotates in the second rotational direction, the second projection engages the protrusion of the counter drive member such that the counter drive member follows the rotation of the detachable member in the second rotational direction.

50. The assembly according claim 49,
wherein the protrusion of the counter drive member is inclined relative to an azimuthal direction.

51. The assembly according to claim 4,
wherein the first and the second projection of the detachable member are spaced apart in an azimuthal direction and in an axial direction.

52. The assembly according to claim 49,
wherein the housing comprises a cut-out,
wherein the counter drive member is arranged at least partially inside the housing,
wherein the detachable member is arranged outside of the housing, and
wherein the protrusion of the counter drive member protrudes through the cut-out of the housing.

53. The assembly according to claim 47,
wherein, during the detachment of detachable member from the housing, the detachable member is configured to carry out a detachment movement comprising a first part wherein the detachable member rotates in the first rotational direction relative to the housing and a subsequent second part wherein the detachable member rotates in the first rotational direction relative to the housing,
wherein the detachable member and the counter drive member are configured such that, during detachment of the detachable member from the housing, the counter drive member follows only the first part of the detachment movement of the detachable member.

54. The assembly according to claim 47,
wherein, during the attachment of detachable member to the housing, the detachable member is configured to carry out an attachment movement comprising a first part wherein the detachable member rotates in the second rotational direction relative to the housing and a subsequent second part wherein the detachable member rotates in the second rotational direction relative to the housing,
wherein the detachable member and the counter drive member are configured such that, during attachment of the detachable member to the housing, the counter drive member follows only the second part of the attachment movement of the detachable member.

55. The assembly according to claim 47,
wherein the counter drive member comprises a spring element biasing the counter drive member in an axial direction.

56. The assembly according to claim 47,
wherein the counter drive member comprises a slot in an axial direction.

57. The assembly according to claim 47,
further comprising a counting member having indicia which correspond to the counted number,
wherein the counting member is configured to follow a rotation of the counter drive member in the second rotational direction relative to the housing, and
wherein the counting member is prevented from following a rotation of the counter drive member in the first rotational direction relative to the housing.

58. The assembly according to claim 27,
wherein the counting member is constrained such that it can only carry out a helical movement relative to the housing.

59. The assembly according to claim 57,
wherein the counting member is arranged inside the housing,
wherein the housing comprises an inner surface having axially extending ribs,
wherein the counting member comprises a claw being engaged with the axially extending ribs, and
wherein the axially extending ribs of the housing and the claw of the counting member are configured to allow a rotation of the counting member in the second rotational direction relative to the housing and to prevent a rotation of the counting member in the first rotational direction relative to the housing.

60. Drug delivery device comprising an assembly according to claim 31.
